(19) United States
(12) Patent Application Publication
(10) Pub. No.: US 2023/0024898 A1
(43) Pub. Date: Jan. 26, 2023

(54) IMMUNOME WIDE ASSOCIATION STUDIES TO IDENTIFY CONDITION-SPECIFIC ANTIGENS

(71) Applicant: Serimmune Inc., Goleta, CA (US)

(72) Inventors: John Shon, Portola Valley, CA (US); Winston A. Haynes, Santa Barbara, CA (US); Patrick Sean Daugherty, Goleta, CA (US)

(21) Appl. No.: 17/555,216

(22) Filed: Dec. 17, 2021

Related U.S. Application Data
(63) Continuation of application No. PCT/US2020/038856, filed on Jun. 20, 2020.
(60) Provisional application No. 62/864,909, filed on Jun. 21, 2019.

(51) Int. Cl. G16B 30/00; G16B 20/30; G16B 40/20; G16H 70/40

(52) U.S. Cl. CPC G16B 30/00; G16B 20/30; G16B 40/20; G16H 70/40

(57) ABSTRACT

The present invention provides compositions and methods that can be used to identify an antigen or epitope region of an antigen specific for a disease or other condition. Such methods incorporate k-mer binding statistics to serum antibody from condition and control cohort samples to predict the suitability of antigen sequences identified as relevant to the disease or condition as antigen markers. Also disclosed herein are systems for performing the same.

### CROSS-REFERENCE TO RELATED APPLICATIONS

This application is a continuation of International Application No. PCT/US2020/038856, filed Jun. 20, 2020, which claims the benefit of U.S. Provisional Application No. 62/864,909 filed Jun. 21, 2019, the contents of which are each hereby incorporated by reference in their entirety.

### SEQUENCE LISTING

The instant application contains a Sequence Listing which has been submitted electronically in ASCII format and is hereby incorporated by reference in its entirety. Said ASCII copy, created on Dec. 17, 2021, is named SUI-005WO-SL As Filed.TXT and is 1,249 bytes in size.

### BACKGROUND

Antibodies present in human specimens serve as the primary analyte and disease biomarker for a large and broad group of infectious, bacterial, viral, allergic, parasitic, and autoimmune diseases. As such, hundreds of distinct antibody detecting tests (collectively referred to as “immunoassays”, have been developed to diagnose human disease using tissue samples that include but are not limited to whole blood, serum, plasma, saliva, urine, and tissue aspirates. Immunoassays remain essential to the diagnosis of autoimmune diseases including, but not limited to, Grave's disease, Sjogren's syndrome Celiac disease, Crohn's disease, Rheumatoid arthritis. Immunoassays are also widely used to diagnose infectious diseases including viral infections (e.g. HIV, Hepatitis C, HSV-1, Zika virus, Epstein Barr virus, and others), bacterial infections (e.g. Streptococcus sp., Helicobacter pylori, Borrellia burdorferi (Lyme), and others), fungal infections (e.g. Valley Fever), and parasitic infections (e.g., Trypanosoma cruzi, Toxoplasma gondii, Taenia solium, Toxocara canis, and others). Furthermore, Immunoassays are often used to identify and monitor allergies (e.g. peanut allergy, milk, pollen, and others. Beyond these areas, immunoassays have demonstrated utility for the diagnosis of neurodegenerative disease, cardiovascular disease, and cancers.

Present methods used to develop diagnostic immunoassays limit the overall sensitivity and specificity that can be obtained from the assay, and thus the utility, because they include extraneous antigen matter (i.e., large proteins, peptides, lipids, whole cell lysates) that can result in cross-reactive binding from unrelated antibodies. Thus, there is an unmet need for diagnostic technologies that can identify and present only those antigen components or set of components that are most specifically recognized by the immune response in individuals with a given phenotype.

More recently, technologies such as protein and peptide arrays have also been developed that attempt to isolate the antigens and epitopes of interest for a given condition. These approaches have had only limited success as 1) the antigens need to be known in advance in order to be placed on the array and 2) the proteins or peptides may be non-specific, as the longer the peptide, the more surface available for binding of non-specific, non-target antibodies. The assays are time consuming and cumbersome, are not amenable to high-throughput analysis, and the data obtained from the wet lab assay are often limited to the specific disease or condition of interest.

Furthermore, in the context of autoimmunity and cancer, if a signal is heterogeneously dispersed across multiple peptides of a single shared antigen, then the “signal” across an antigen will not rise significantly above noise if a given epitope is not shared across the cohort of interest. Thus, methods that attempt to look at “signal” across a set of disease samples vs. a set of control samples will fail to identify a specific epitope if it is not shared, and the antigen will also potentially not be recognized as a shared antigen due to the heterogeneity of epitopes present on the antigen for a given array.

What is needed, therefore, are methods for performing a single assay on a sera sample that can be used for analysis of all possible combination of antigen sequences corresponding to any disease or condition to determine a state of an individual, which can be used, e.g., for diagnosis, prediction of treatment therapy, or identification of a therapeutic target. Furthermore, these methods should not be limited to identification of a specific epitope in identifying antigens.

### SUMMARY

Provided herein, according to some embodiments, is a method of identifying an antigen marker for a condition, the method comprising: identifying a condition cohort and a control cohort for comparison; providing a set of antigens corresponding to said condition, wherein the sequence of each antigen is tiled into subsequences; providing an enrichment score for each of said subsequences for both said condition cohort and said control cohort; for each antigen in said set of antigens: determining an antigenic score of said antigen for said condition cohort and said control cohort from said enrichment scores for subsequences within said antigen, and comparing said antigenic score for said condition cohort and said control cohort to determine an antigen outlier score; and identifying said antigen as an antigen marker for said condition if said antigen outlier score exceeds a threshold value.

In some embodiments, the enrichment score is determined from a motif enrichment score determined for a motif comprising said subsequence. In some embodiments, the enrichment score is determined from identification of relative binding of subsequences to antibodies from a serum sample between said condition cohort and said control cohort. In some embodiments, the method further comprises determining said enrichment score by identifying relative binding of subsequences to antibodies from a serum sample between said condition cohort and said control cohort.

In some embodiments, the antigenic score is determined from the highest subsequence enrichment score for said antigen sequence in said cohort. In some embodiments, the antigenic score is determined from the sum of all subsequence enrichment scores for said antigen sequence in said cohort. In some embodiments, the antigenic score is determined from the highest average value of subsequence enrichment scores within a window of n subsequences for said antigen sequence in said cohort. In some embodiments, the antigenic score is determined from the sum of n maximum subsequence enrichment scores across the antigen sequence.

In some embodiments, the comparing said antigenic score for said condition cohort and said control cohort comprises calculating a statistical difference between antigenic scores from said sample cohort and said control cohort for said antigen. In some embodiments, the threshold value represents a statistical difference sufficient for identifying said antigen as an antigen marker. In some embodiments, the statistical difference is determined from a statistical analysis selected from the group consisting of: Cohen's d effect size, Mann-Whitney U p-value, Kolmogorov-Smirnov p-value, and Outlier sum. In some embodiments, the statistical difference comprises a correction for multiple hypothesis testing. In some embodiments, the correction is Bonferroni correction or false discovery rate. In some embodiments, the threshold is determined from a ranking of antigen outlier scores determined from said set of antigens.

In some embodiments, the subsequences are k-mers. In some embodiments, the k-mers comprise 5-mers, 6-mers, 7-mers, 8-mers, 9-mers, or 10-mers. In some embodiments, the subsequence comprises a k-mer sequence with at least k-n defined amino acid positions, wherein k is 8, 9 or 10, and wherein n is 2, 3, 4, 5, or 6.

In some embodiments, the antigen sequences are amino acid sequences. In some embodiments, the antigen marker comprises a protein, a RNA, or an aptamer.

In some embodiments, the condition cohort comprises one or more samples from one or more patients, wherein said patients have been diagnosed with an infection, an autoimmune disorder, a cancer, a neurological disorder, or a chronic disease, or wherein said patient has been administered a therapeutic agent or a vaccine.

In some embodiments, providing said enrichment score comprises: contacting a display system comprising a plurality of distinct peptides with a biological sample comprising a plurality of antibodies, wherein the plurality of antibodies is known or suspected to comprise antibodies for said condition, and wherein the contacting is performed under conditions sufficient for the specimen antibodies to specifically bind to a cognate epitope on said plurality of distinct peptides; measuring the binding between the plurality of distinct peptides and the specimen antibodies; and identifying an enrichment score for said subsequence from the amount of binding measured for said subsequence.

In some embodiments, the peptides are randomly generated. In some embodiments, the peptides are from 8-mer to 15-mer peptides. In some embodiments, the peptides are 12-mer peptides. In some embodiments, the display system comprising at least 10, at least 100, at least 1000, at least 10⁴, at least 10⁵, at least 10⁶, at least 10⁷, or at least 10⁸ distinct peptides. In some embodiments, the said peptides are 12-mer peptides and are randomly generated.

In some embodiments, the determination of said antigenic score and said antigenic outlier score is implemented as a set of computer program instructions stored on a non-transitory computer readable storage medium for execution by a processor of a computer system. In some embodiments, the identifying said antigen as an antigen marker for said condition if said antigen outlier score exceeds a threshold value is implemented as a set of computer program instructions stored on a non-transitory computer readable storage medium for execution by a processor of a computer system.

Also provided herein, according to some embodiments, is a method of identifying one or more antigenic epitopes on an antigen marker specific for a condition cohort as compared to a control cohort, the method comprising: identifying a condition cohort and a control cohort for comparison; providing an antigen corresponding to said condition, wherein the sequence of said antigen is tiled into subsequences; providing an enrichment score for each of said subsequences for samples from both said condition cohort and said control cohort; determining a statistical difference between enrichment scores in one or more regions of said antigen for said samples from said condition cohort compared to said samples from said control cohort; and identifying said one or more regions as an antigenic epitope specific for said condition cohort as compared to said control cohort if said statistical difference exceeds a threshold value.

In some embodiments, the enrichment score is determined from a motif enrichment score determined for a motif comprising said subsequence. In some embodiments, the enrichment score is determined from identification of relative binding of subsequences to antibodies from a serum sample between said condition cohort and said control cohort. In some embodiments, the method further comprises determining said enrichment score by identifying relative binding of subsequences to antibodies from a serum sample between said condition cohort and said control cohort.

In some embodiments, the comparing said enrichment score for said condition cohort and said control cohort comprises calculating a statistical difference between enrichment scores from said sample cohort and said control cohort for said antigen. In some embodiments, the threshold value represents a statistical difference sufficient for identifying said one or more regions as an antigenic epitope. In some embodiments, the statistical difference is determined from a statistical analysis selected from the group consisting of: Cohen's d effect size, Mann-Whitney U p-value, Kolmogorov-Smirnov p-value, and Outlier sum. In some embodiments, the statistical difference comprises a correction for multiple hypothesis testing. In some embodiments, the correction is Bonferroni correction or false discovery rate.

In some embodiments, the subsequences are k-mers. In some embodiments, the k-mers comprise 5-mers, 6-mers, 7-mers, 8-mers, 9-mers, or 10-mers. In some embodiments, the subsequence comprises a k-mer sequence with at least k-n defined amino acid positions, wherein k is 8, 9 or 10, and wherein n is 2, 3, 4, 5, or 6.

In some embodiments, the antigen sequences are amino acid sequences. In some embodiments, the antigen marker comprises a protein, a RNA, or an aptamer.

In some embodiments, the condition cohort comprises one or more samples from one or more patients, wherein said patients have been diagnosed with an infection, an autoimmune disorder, a cancer, a neurological disorder, or a chronic disease, or wherein said patient has been administered a therapeutic agent or a vaccine.

In some embodiments, providing said enrichment score comprises: contacting a display system comprising a plurality of distinct peptides with a biological sample comprising a plurality of antibodies, wherein the plurality of antibodies is known or suspected to comprise antibodies for said condition, and wherein the contacting is performed under conditions sufficient for the specimen antibodies to specifically bind to a cognate epitope on said plurality of distinct peptides; measuring the binding between the plurality of distinct peptides and the specimen antibodies; and identifying an enrichment score for said subsequence from the amount of binding measured for said subsequence.

In some embodiments, the peptides are randomly generated. In some embodiments, the peptides are from 8-mer to 15-mer peptides. In some embodiments, the peptides are 12-mer peptides. In some embodiments, the display system comprising at least 10, at least 100, at least 1000, at least 10⁴, at least 10⁵, at least 10⁶, at least 10⁷, or at least 10⁸ distinct peptides. In some embodiments, the peptides are 12-mer peptides and are randomly generated.

In some embodiments, determining a statistical difference between enrichment scores in one or more regions of said antigen for said samples from said condition cohort compared to said samples from said control cohort is implemented as a set of computer program instructions stored on a non-transitory computer readable storage medium for execution by a processor of a computer system. In some embodiments, identifying said one or more regions as an antigenic epitope specific for said condition cohort as compared to said control cohort if said statistical difference exceeds a threshold value is implemented as a set of computer program instructions stored on a non-transitory computer readable storage medium for execution by a processor of a computer system.

Also provided herein, according to some embodiments, is a method of identifying a protein marker for a condition, the method comprising: identifying a condition cohort and a control cohort for comparison; providing a set of proteins from a proteome corresponding to said condition, wherein said proteins are tiled into k-mer sequences; providing an enrichment score for said plurality of k-mer sequences from serum samples from subjects having said condition phenotype and subjects having said control phenotype, wherein said enrichment score is determined from measuring a level of binding of said k-mer sequence to antibodies in each serum sample; for each protein in said set of proteins: determining an antigenic score of said protein for said condition cohort and said control cohort from said enrichment scores for k-mer sequences within said protein, and comparing said antigenic score for said condition cohort and said control cohort to determine a protein outlier score; and identifying said protein as a protein marker for said condition if said protein outlier score exceeds a threshold value.

Also provided herein, according to some embodiments, is a system for identifying an antigen marker for a condition comprising a non-transitory computer readable storage medium and a processor, said storage medium comprising: enrichment scores for subsequences of antigens corresponding to said condition, said enrichment scores specific to a condition cohort and a control cohort; instructions for generating an antigenic score of each antigen specific to said condition cohort and said control cohort from said enrichment scores of subsequences of said antigen; and instructions for generating an antigenic outlier score by comparing the statistical difference between said antigenic score for said antigen specific for said condition cohort and said control cohort.

In some embodiments, the system further comprises instructions for generating an output identifying antigens suitable as an antigen marker for said condition based on said antigen outlier score. In some embodiments, the system further comprises instructions for receiving sequences of said antigen corresponding to said condition. In some embodiments, the system further comprises instructions for tiling sequences of said antigens corresponding to said condition into subsequences. In some embodiments, the system further comprises instructions for receiving an enrichment score for said subsequences.

Also provided herein, according to some embodiments, is a system for identifying one or more antigenic epitopes on an antigen marker specific for a condition cohort comprising a non-transitory computer readable storage medium and a processor, said storage medium comprising: enrichment scores for subsequences of said antigenic marker, said enrichment scores specific to a condition cohort and a control cohort; and instructions for determining a statistical difference between enrichment scores in one or more regions of said antigen for said samples from said condition cohort compared to said samples from said control cohort.

In some embodiments, the system further comprises instructions for generating an output identifying said one or more regions as an antigenic epitope specific for said condition cohort as compared to said control cohort if said statistical difference exceeds a threshold value. In some embodiments, the system further comprises instructions for receiving sequences of said antigen corresponding to said condition. In some embodiments, the system further comprises instructions for tiling sequences of said antigens corresponding to said condition into subsequences. In some embodiments, the system further comprises instructions for receiving an enrichment score for said subsequences.

### BRIEF DESCRIPTION OF THE DRAWINGS

The foregoing and other objects, features and advantages will be apparent from the following description of particular embodiments of the invention, as illustrated in the accompanying drawings in which like reference characters refer to the same parts throughout the different views. The drawings are not necessarily to scale, emphasis instead placed upon illustrating the principles of various embodiments of the invention.

FIG. 1 shows values of enrichment scores for each tiled k-mer subsequence (at its respective amino acid position) of a protein.

FIG. 2 and FIG. 3 show the location and maximum enrichment score (dot) for a k-mer from the tiled scores for the protein as provided in FIG. 1.

FIG. 4 shows the maximum score (used as an enrichment score) determined as shown in FIG. 1-3 for individual proteins across a number of proteins taken from multiple samples from each cohort.

FIG. 5 illustrates sample rankings of antigens identified using the methods described herein comparing statistical difference of antigenic score between condition and control cohorts.

FIG. 6 shows a comparison of antigenic scores for validated antigen NY-ESO-1 in sample sera from melanoma patients as determined by traditional enzyme linked immunosorbant assays (ELISA) vs. as determined by the generation of an antigenic score via k-mer subsequence analysis disclosed herein.

FIG. 7 shows a plot of k-mer subsequence maximum score for NY_ESO-1 from each of a plurality of samples from cancer and non-cancer cohorts.

FIG. 8 shows epitope-level resolution of antigenicity for NY-ESO-1 using tiled k-mer sequences and k-mer enrichment values from sera of patients i) responsive to therapy and ii) not responsive to therapy both before (‘Baseline’) and after therapy (‘On Therapy’, approximately 3 months after treatment).

FIG. 9 illustrates rankings of antigens as biomarkers for Sjogren's patients as identified using the methods described herein comparing statistical difference of antigenic score between condition and control cohorts.

FIG. 10 shows a plot of k-mer subsequence maximum score for SSB antigen from each of a plurality of samples from control, Sjogren's SSB−, and Sjogren's SSB+ cohorts.

FIG. 11 shows a comparison of antigenic scores for validated antigen CENPA in sample sera from Sjogren's patients as determined by traditional enzyme linked immunosorbant assays (ELISA) vs. as determined by the generation of an antigenic score via k-mer subsequence analysis disclosed herein.

FIG. 12 illustrates rankings of antigens as biomarkers for natural HSV2 infection as compared to the HSV2 vaccination using the methods described herein comparing statistical difference of antigenic score between condition and control cohorts.

FIG. 13 provides a chart showing maximum k-mer enrichment values identified on envelope glycoprotein E for serum samples from HSV2 infected patients (‘Case’) (i.e., condition) and HSV2 vaccinated patients (‘Control’).

FIG. 14 shows a plot of k-mer subsequence maximum scores for Envelope Glycoprotein E from each of a plurality of samples from sera from HSV2 infected patients (‘Case’) (i.e., condition) and HSV2 vaccinated patients (‘Control’).

FIG. 15 shows a plot of k-mer subsequence maximum score for Envelope Glycoprotein D from each of a plurality of samples from sera from HSV2 infected patients (‘Case’) (i.e., condition) and HSV2 vaccinated patients (‘Control’).

### DETAILED DESCRIPTION

The details of various embodiments of the invention are set forth in the description below. Other features, objects, and advantages of the invention will be apparent from the description and the drawings, and from the claims.

### Introduction

In a given disease state, the immune system forms antibodies against antigens that appear to be foreign or “non-self”. For infections, these antigens, and epitopes in these antigens tend to be conserved across a population. While methods have previously been successful identifying shared epitopes/motifs in the context of infectious disease, signal in both cancer and autoimmunity has been difficult to detect due to heterogeneity in epitopes observed. However, as described herein, conserved antigens that correspond to a disease state do not require conserved epitopes on a given antigen.

We have developed the SERA assay which uses an extremely large (10¹⁰) random bacterial display library to capture and decode epitope information in sera using NGS and computational methods.

Provided herein are methods and compositions that use information corresponding to that obtained from the SERA assay and databases of antigenic information for peptides developed from SERA in combination with proteomic information to identify shared antigens. This method is used to identify the most significant shared antigens, including those with signals that do not present shared epitopes. Thus, provided herein, according to some embodiments, is a method that identifies such shared antigens and additionally provides epitope level resolution to reactivity against the shared antigens

To identify shared antigen signals, we break every protein into constituent subsequences and calculate an antigenicity signal for each sample across the set of subsequences. We then compare the sample signals for each protein between the disease and control cohorts, identifying proteins with differential antigenicity between the cohorts.

By removing the constraint that samples must share specific epitope sequences, we identify antigens that were undetected by existing computational solutions. This has shown substantial benefit in application to cancer and autoimmunity projects where epitopes may be private but antigens are shared. Peptide arrays that look at shared signal in disease vs. control in single addresses will have diluted signal that will not rise above noise if there is insufficient sharing of those addresses.

The method simultaneously provides antigen- and epitope-level resolution at very high-throughput, which is not feasible using other wet lab technologies

When we use SERA to provide antigenic signal from a random library for each sample, the method does not rely on including an antigen or set of antigens in an assay prior to analysis. The method works on one antigen up to multiple proteomes scale (>20,000 proteins) with computational efficiency. This scalability allows for data and statistically driven discoveries in large cohorts. Data from large control cohorts improves the specificity of findings.

As one example, in a combined analysis of prostate cancer and melanoma, we identified NY-ESO-1 as the most differentially antigenic protein compared to controls and found that the epitopes contributing to each sample occurred in neighboring, but non-identical, regions of the protein sequence. We then verified that the region we identify as being antigenic is consistent with prior literature that used synthetic peptides to identify the antigenic epitopes of NY-ESO-1.

### Definitions

Terms used in the claims and specification are defined as set forth below unless otherwise specified.

Unless specifically stated or otherwise apparent from context, as used herein the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Numerical values provided herein can sometimes be considered to be modified by the term about, where context makes clear that the ranges encompassed by the modification are consistent with operability of the invention and definiteness of the claims.

The term “enrichment” as used herein, corresponds with the number of observations of a peptide (including protein or antigen subsequences), pattern, or motif, within an epitope repertoire compared with the number expected within a random dataset of equivalent size. This information can be used to generate an “enrichment score” for the peptide, pattern, or motif, which is a measure of the expected relative antigenicity of the peptide, pattern, or motif in a sample sera from a cohort. For example, in a hypothetical 9-mer peptide library, where X is any amino acid, the pattern QPXXPFX[ED] (SEQ ID NO:3) is expected to occur once in every 800,000 ((1aa/20aa)4×(2aa/20aa)×2) random sequences (aa=amino acid). If 4 million sequences were determined, then one would expect to observe five (5) occurrences (i.e., once in every 800,000 sequences). As an example, if the pattern was actually observed in 50 unique peptides sequences (i.e. 50 observations) in an epitope repertoire, then the pattern would be “enriched” by 10-fold versus random. Such a determination of enrichment scores specific to patient samples using peptide display libraries is described in PCT Publication No WO/2017/083874, filed Nov. 14, 2016, “Methods and Compositions for Assessing Antibody Specificities,” (i.e., “the SERA technology”) incorporated herein by reference in its entirety.

The term “antigenic score” as used herein refers to a measure of expected antigenicity of a protein or antigen marker in a sample cohort, such as one or more condition cohorts and/or control cohorts. As described herein, the antigenic score is determined using enrichment scores from k-mer subsequences or motifs in proteins of a condition relevant proteome from the sample.

The term “antigen outlier score” used herein refers to a score generated by comparison of antigenic scores of antigens or proteins between samples and/or cohorts to identify whether an antigen is useful as an antigen marker. Such cohorts can be relevant to biomarkers of disease or biomarkers of treatment response, such as those having or not having the condition before or after treatment, or at a certain defined stage of the disease before or/after treatment. In some embodiments, identification of whether an antigen or protein is useful as an antigen marker for at least one of the cohorts comprises identifying whether the antigen outlier score for an antigen or protein is above a predetermined threshold. Such a threshold can be set to identify a statistically significant antigen marker for a condition, i.e., can be used to distinguish between a sample from a condition and control (i.e., reference) cohort.

The term “threshold” as used herein refers to the magnitude or intensity that must be exceeded for a certain reaction, phenomenon, result, or condition to occur or be considered relevant. For example, the threshold can be a numerical value above which an antigenic score is considered relevant. The relevance can depend on context, e.g., it may refer to a positive, reactive or statistically significant relevance.

As used here, the term “next generation sequencing” (NGS) and the like is used to refer to high throughput nucleic acid sequencing (HTS) approaches. Platforms for NGS that rely on different sequencing technologies are commercially available from a number of vendors such as Pacific Biosciences, Ion Torrent from Thermo Fisher, 454 Life Sciences, Illumina, Inc. (e.g., MiSeq, NextSeq, HiSeq) and Oxford Nanopore. For a review of NGS technologies, see, e.g., van Dijk E L et al. Ten years of next-generation sequencing technology. Trends Genet. 2014 September; 30(9):418-26, herein incorporated by reference in its entirety for all purposes.

The term “surface display” as used herein refers to the presentation of heterologous peptides and proteins on an array surface, such as the outer surface of a biological particle such as a living cell, virus, or bacteriophage.

As used herein, a “library of peptides” or a “peptide library” refers to a collection of a peptide fragments typically used for screening purposes. The terms “peptide,” “polypeptide,” “amino acid sequence,” “peptide sequence,” and “protein” are used interchangeably to refer to two or more amino acids linked together and imply no particular length. Amino acids and peptides can be naturally occurring or synthetic (e.g., unnatural amino acids or amino acid analogs). Amino acids and peptides can also comprise, or be further modified to comprise, reactive groups, such as reactive groups for attaching amino acids or peptides to solid substrates, reactive groups for labeling amino acids or peptides, or reactive groups for attaching other moieties of interest to amino acids or peptides. Reactive groups include, but are not limited to, chemically-reactive groups such as reactive thiols (e.g., maleimide based reactive groups), reactive amines (e.g., N-hydroxysuccinimide based reactive groups), “click chemistry” groups (e.g., reactive alkyne groups), and aldehydes bearing formylglycine (FGly).

The term “disease” refers to an abnormal condition affecting the body of an organism. The term “disorder” refers to a functional abnormality or disturbance. The terms disease or disorder are used interchangeably herein unless otherwise noted or clear given the context in which the term is used. The terms disease and disorder may also be referred to collectively as a “condition.”

The term “phenotype” as used herein comprises the composite of an organism's observable characteristics or traits, such as its morphology, development, biochemical or physiological properties, phenology, behavior, and products of behavior.

The term percent “identity,” in the context of two or more nucleic acid or polypeptide sequences, refer to two or more sequences or subsequences that have a specified percentage of nucleotides or amino acid residues that are the same, when compared and aligned for maximum correspondence, as measured using one of the sequence comparison algorithms described below (e.g., BLASTP and BLASTN or other algorithms available to persons of skill) or by visual inspection. Depending on the application, the percent “identity” can exist over a region of the sequence being compared, e.g., over a functional domain, or, alternatively, exist over the full length of the two sequences to be compared.

For sequence comparison, typically one sequence acts as a reference sequence to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al., infra).

One example of an algorithm that is suitable for determining percent sequence identity and sequence similarity is the BLAST algorithm, which is described in Altschul et al., J. Mol. Biol. 215:403-410 (1990). Software for performing BLAST analyses is publicly available through the National Center for Biotechnology Information (www.ncbi.nlm.nih.gov/).

The term “sufficient amount” means an amount sufficient to produce a desired effect.

The term “therapeutically effective amount” is an amount that is effective to ameliorate a symptom of a disease. In some contexts, a therapeutically effective amount can be a “prophylactically effective amount” as prophylaxis can be considered therapy, provided such interpretation does not adversely impact any determination of the validity of any claim for any reason.

### Antigen Discovery

The present invention provides methods and compositions to identify disease-specific, proteome-based, antigenic signals. The identified antigens can be used as potential markers of disease or markers of therapeutic response. The identified antigens can also be used as potential therapeutic targets.

In brief, as described herein, methods of identifying disease-specific antigens comprise, for example, i) identifying or determining an antigenic response of sera from a disease state and a comparison control state against a defined set of k-mer peptides, ii) using this response to predict an antigenic response of an antigen comprising one or more k-mers to the disease sera and the control sera, and iii) determining if the difference between the antigenic response to the disease sera vs. the control sera exceeds a threshold to identify the antigen as useful for providing a disease-specific, proteome-based, antigenic signal. In some embodiments, a proteome corresponding to the disease-state is identified and protein sequences from this proteome are broken into constituent k-mer sequences for identification of antigenic response to each protein by the disease sera and the control sera.

In some embodiments, for each protein and sample (e.g., disease serum and control serum), the strongest, linear antigen (k-mer) is identified. In some embodiments, for every protein, the antigenic signals between the disease and control populations (i.e., disease and control sera) are compared. In some embodiments, the proteins with the strongest antigenic signal are identified for the disease cohort.

Provided below are steps describing the discovery and identification of antigenic antigens that can be used to distinguish sera from a disease state vs. a control or non-disease state, according to some embodiments of the invention.

Enrichment Scores for k-Mer Subsequences

Initially, we identify samples that will be utilized as the condition and control cohorts. For each sample, we will identify or determine the k-mer level statistics for all k-mers in the protein database.

In some embodiments, as described herein, this data is derived from patient samples using peptide display libraries as describe in PCT Publication No WO/2017/083874, filed Nov. 14, 2016, “Methods and Compositions for Assessing Antibody Specificities,” (i.e., “the SERA technology”) incorporated herein by reference in its entirety. In some embodiments, SERA uses bacterial display technology to present a diverse set of 12mer peptides to serum antibodies. Peptides that bind to serum antibodies are separated using magnetic beads and sequenced using next generation sequencing. Each 12mer is broken into kmer components and log-enrichments of these kmers are calculated, where enrichment indicates the number of observations compared to expectation based on expected frequency based on kmer population statistics in the random 12mer peptides. This is performed for each sample from each cohort to identify sample-specific and cohort-specific k-mer enrichment scores.

The current methodology described herein was derived using the SERA technology, but would be applicable to any technology generating epitope level data (e.g., peptide arrays and other sequencing based approaches). Thus, determination of antigenic peptide sequences is not limited to the above method, and can be determined using any other peptide driven technologies.

Identification of k-Mer Sequences Relevant to Condition

For the disease-state of interest, a proteome relevant to the condition cohort is obtained. Such proteomes (e.g., human proteome or infectious agent proteome) can be obtained from publicly available sequence databases (e.g., Uniprot). For brevity, we will these amino acid sequences are referred to as “proteins”, but this approach could be applied to non-protein antigen sequences.

Each protein is tiled into constitutive k-mers that each represent a consecutive sequence of k amino acids. In preferred embodiments, k is one or a combination of 5, 6, or 7. For example, the protein sequence ABCDEFG would be broken into the tiled 5mers ABCDE, BCDEF, CDEFG.

Enrichment scores for each k-mer sequence of a protein specific to a sample and/or cohort are used to identify an antigenic score for the protein in a sample and/or cohort. First, a k-mer level enrichment score is determined or identified. This value corresponds with the binding of sera from a sample to the k-mer as compared to the expectation for the number of observations for a particular k-mer. In some embodiments, the k-mer level enrichment value is based on a ‘comparison’ of the number of standard deviations a particular enrichment value is from the enrichments of a control cohort, where these controls may either be the comparison cohort or a third cohort. Although k-mer enrichment scores described herein are determined based on relative enrichment or number of standard deviations, different values for each k-mer enrichment score can also be used, including raw counts or alternative normalization approaches.

In some embodiments, k-mer enrichment scores are determined for a k-mer motif, instead of a specific sequence. A set of k-mer sequences related to the k-mer present in the antigen may constitute a “motif”, in which some positions in the sequence may have multiple amino acids possible in the position. Motif scores aggregate the constituent k-mer enrichment scores and may be also be used for the k-mer enrichment score.

Antigenic Score for a Protein

An antigenic score is identified for proteins in a proteome relevant to the condition of interest. This score corresponds with the specificity of antigenicity of each protein with r respect to the condition of interest (i.e., in a sample cohort as compared to a control cohort). Enrichment scores specific to each sample and/or cohort for each k-mer subsequence within each protein are used to determine an antigenic score for each protein specific to each sample and/or cohort (e.g., disease and control). Several methods to determine antigenic scores from k-mer enrichment scores are disclosed herein.

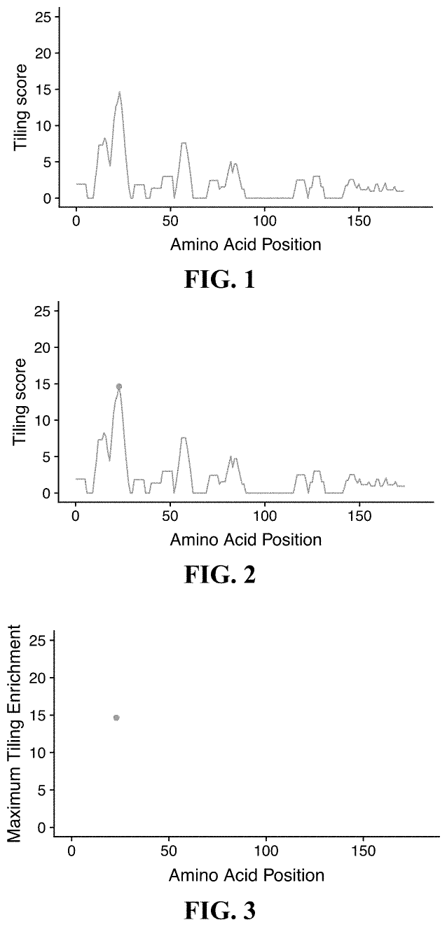

In some embodiments, determining an antigenic score from the k-mer enrichment scores comprises tiling k-mer sequences in a protein (or other non-protein antigen sequence) in a relevant proteome of the sample as shown in FIG. 1. In some embodiments, this k-mer level statistic is smoothed (i.e. averaged) across a window of a number k-mers (e.g., a window of 5 k-mers). In some embodiments, multiple k-mer enrichment score are used (e.g., simultaneously using 5mers and 6mers), and the scores are determined from the sum across the k-mer enrichment scores.

In some embodiments, the maximum k-mer enrichment score for a protein is used to determine the antigenic score for that protein. Shown in FIGS. 2 and 3 are the location and maximum score for a k-mer antigenic signal from the tiled scores for the protein as provided in FIG. 1. In another embodiment, the sum of the n maximum k-mer enrichment scores across the protein, where n could include one or more k-mer enrichment score peaks along a tiled protein sequence, is used. In another embodiment, the summed score of all k-mer enrichment scores in the protein is used.

Antigen Outlier Score to Identify a Condition-Specific Antigen

Antigenic scores for each protein as determined above are compared between cohorts. A statistical significance of the difference of antigenic scores for each protein between cohorts is calculated. The statistical difference between the antigenic scores of the cohorts is used to determine an antigen outlier score, which is a measure of the protein's predicted antigenic specificity in a cohort. In some embodiments, comparison of the condition and control cohorts is done with one of the following statistical methods: 1. Effect size (defined as Cohen's d effect size), 2. Mann-Whitney U p-value, 3. Kolmogorov-Smirnov p-value, and 4. Outlier sum (described in https://www.ncbi.nlm.nih.gov/pubmed/16702229). For Mann-Whitney U Statistics, signals are identified based on shifts across a population (non-parametric, rank order). P-value is based on established distributions. For Outlier Sum, signals are identified as “outliers” in a meaningful subset of the population. P-value is based on permutations and Central Limit Theorem. Other suitable statistical methods known to those of skill in the art can be used. In some embodiments, these statistical analyses can be corrected for multiple hypothesis testing using an approach like the Bonferroni correction or the false discovery rate.

The current methods being used to statistically compare the antigenic scores to determine an antigen outlier score for an antigen are detailed above, but there are potentially innumerably many alternative statistical tests that might be applied to compare the condition and control cohorts (i.e. T-test, COPA outlier, chi-squared test). In particular, a significant signal may appear in a given antigen in multiple places for a given sample, methods to improve the total antigenic signal could be employed

Each protein or antigen is labeled as a relevant antigen if the difference between cohorts exceeds a threshold value. In some embodiments, we generate a ranking of proteins based on the statistics used to compare condition and control cohorts. Sample rankings generated by our method are provided in FIG. 5.

In some embodiments, proteins or antigens identified as relevant to the condition could be used to: i) develop a diagnostic, e.g., an ELISA or SERA panel, ii) identify a therapeutic target for monoclonal antibodies, and iii) identify a vaccine target.

Epitope Identification

For each sample, we label the height and location of the maximum value for the k-mer enrichment score along the sequence of a protein. Thus, for each protein, our method provides epitope resolution for antigenic regions.

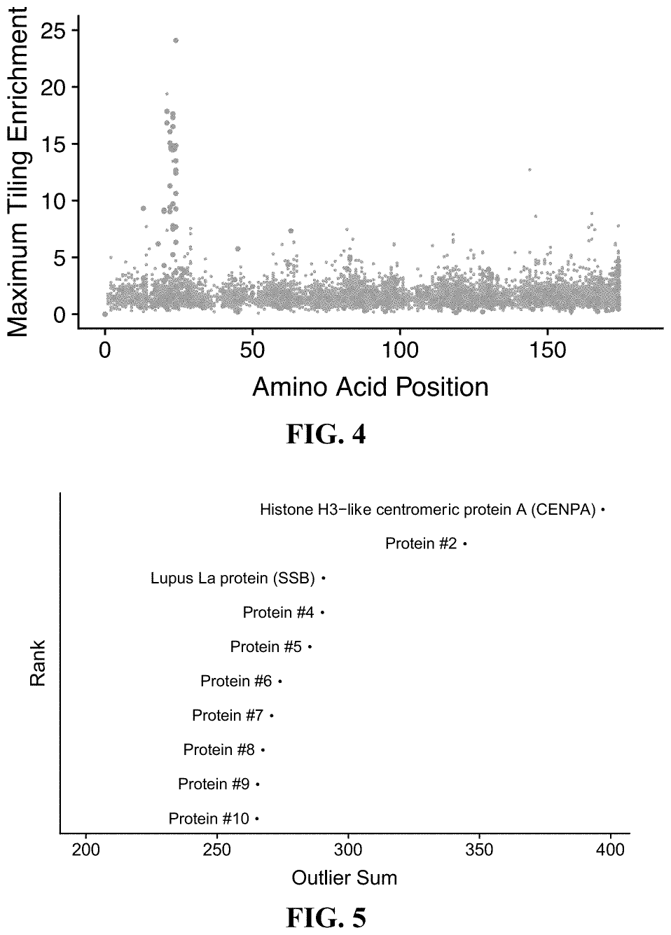

As an example, maximum k-mer enrichment scores for the protein from FIG. 1-3 for each sample and each cohort are determined and overlapped as shown in FIG. 4. Maximum k-mer scores from sera from disease samples are shown in red. Maximum k-mer enrichment scores from sera for control samples are shown in green. A cluster of high k-mer enrichment scores is shown around position 20-25 from samples for disease sera only. This method therefore provides both identification of a disease-specific antigen, as well as identification of the location of the disease-specific epitope on the identified antigen.

Example of Score Determination

In some embodiments, the identification of antigens specific to a condition as described herein can be specifically identified as described below:

We define condition (7), control (U), and (optionally) third control (V) cohorts of samples. We begin with 12mer amino acid sequences for each sample generated by the Serimmune Epitope Repertoire Analysis pipeline.

Enrichment Score Calculation

For each 12mer, we break it into constituent k-mers (where k=5 and k=6). For every k-mer in each sample (S), we calculate enrichment as:

Eₛ(kmer)=n_{S}(kmer)/e_{S}(kmer)

where n(k-mer) is the number of unique 12mers containing a particular k-mer and e_{S}(kmer) is the expected number of k-mer reads for the sample, defined as:

$${e}_{S} ( kmer ) = {N}_{S} ( {L}_{seq} - k + 1 ) {\prod }_{i = 1}^{k} {p}_{i}$$

where N_{S} is the number of 12mer reads generated for 5, L_{seq} is the length of the amino acid reads (12), k is the k-mer length, and pᵢ is the amino acid proportion for the ith amino acid in k-mer in all 12mers from S.

For every k-mer, we normalize enrichment values to a control population. We define the control enrichment values as:

C={Eᵥ(kmer):w∈W}

where W is the third control cohort (V, if defined), otherwise the control cohort (U) is used.

The normalized enrichment is calculated as:

$${F}_{S} ( kmer ) = \frac{{E}_{S} ( kmer ) - μ ( C )}{σ ( C )}$$

where μ(C) is the mean of C and σ(C) is the standard deviation of C.

Antigenic Score Calculation.

For each protein p and sample s, we calculate an antigenic score P(s,p), defined as:

$$P ( s , p ) = {max}_{1 \leq i \leq len ( p )} {\sum }_{k = 5}^{6} {\sum }_{j = i}^{min ( i + w , len ( p ) - k )} {G}_{S} ( kmer ( j , k , p ) )$$

where w is the width of the smoothing window, len(p) is the length of protein p, k-mer(j,k,p) is the k-mer of length k at location j in protein p, and G_{S} is either E_{S} or F_{S}.

Similarly, we record the location of this maximum statistics value, P_{loc}(s, p) as:

$${P}_{loc} ( s , p ) = {arg max}_{1 \leq i \leq len ( p )} {\sum }_{k = 5}^{6} {\sum }_{j = i}^{min ( i + w , len ( p ) - k )} {G}_{S} ( kmer ( j , k , p ) )$$

Cohort Comparison Statistics and Antigen Outlier Score

For each protein p, we define our condition enrichments as:

A(p)={P(t,p): t∈T}

Similarly, we define our control enrichments as:

B(p)={P(u,p):u∈U}

We use a variety of statistical tests to compare A(p) and B(p), including traditional tests like the Mann-Whitney U and Kolmogorov-Smirnov. We calculate effect size as the Hedges' g statistic.

We calculate the Outlier Sum, which we define as O(p), statistic defined in Tibshirani and Hastie, ‘Outlier sums for differential gene expression analysis.’ Biostatistics, 2007. We perform 1,000 random permutations of the samples in A(p) and B(p) and calculate the Outlier Sum to calculate O⁰(p), the null distribution of the Outlier Sum for protein p.

We calculate the z-score as:

$${z}_{O ( p )} = \frac{O ( p ) - {μ}_{{O}^{0} ( p )}}{{σ}_{{O}^{0} ( p )}}$$

Since the Outlier Sum is a sum of i.i.d. variables, we can apply the Central Limit Theorem and calculate a p-value for z_{O(p)} using the normal distribution.

We define the sets of condition and control locations as:

A_{loc}(p)={P_{loc}(t,p):t∈T}

B_{loc}(p)={P_{loc}(u,p):u∈U}

We perform a Kolmogorov-Smirnov test comparing A_{loc}(p) and B_{loc}(p) to identify proteins with locational conservation of epitopes.

### Samples

As used herein, a “sample” refers to any material known to contain or suspected to contain specimen binding molecules (e.g., antibodies). In general, the sample will be a liquid. The sample can be a material that originated as a liquid or can be material processed to be in liquid form. The sample can be the material directly isolated from a source (i.e., untreated) or it can be further processed for use in the method (e.g., diluted, filtered, cell depleted, particulate depleted, assayed, preserved, or other otherwise pre-processed).

Samples include, but are not limited to, serum, blood, saliva, urine, tissue, tissue homogenates, stool, spinal fluid, and lysate derived from animal sources. The sample can include a mixture of different source materials. A sample can be a bodily fluid isolated from any animal that produces or suspected to produce the binding molecule of interest. The animal can be known or suspected of having a disease. The animal can also be known or suspected of having binding molecules that bind antigens or epitopes associated with the disease. In an illustrative non-limiting example, the sample can be processed serum from human suspected to have a specific disease and suspected to produce antibodies that bind epitopes that correlate with the disease. Diseases include, but are not limited to, a bacterial infection, a viral infection, a parasitic infection, an autoimmune disorder, cancer, and an allergy. Disease can also refer to a specific state or progression of a disease, or a state of a disease corresponding to predicted treatment efficacy. Thus a sample from a subject identified as having a disease or condition can include samples from patients diagnosed as having an infection, an autoimmune disorder, a cancer, a neurological disorder, or a chronic disease. In some embodiments, the chronic disease is Chronic Fatigue Syndrome. The sample can also come from a patient that has been administered a therapeutic agent or a vaccine.

Samples from the same identified disease or phenotype can be grouped into a sample cohort. Samples that are negative for the disease or phenotype can be grouped into a control cohort. Closely-related cohorts, such as vaccinated patients vs. infected patients can also be compared using the methods described herein.

As described herein, the compositions and methods of the invention may be used to characterize a phenotype in a sample of interest. The phenotype can be any phenotype of interest that may be characterized using the subject compositions and methods. Consider a non-limiting example wherein the phenotype comprises a disease or disorder. In such cases, the characterizing may be providing a diagnosis, prognosis or theranosis for the disease or disorder. In an illustrative embodiment, a sample from a subject is analyzed using the compositions and methods of the invention. The analysis is then used to predict or determine the presence, stage, grade, outcome, or likely therapeutic response of a disease or disorder in the subject. The analysis can also be used to assist in making such prediction or determination.

The repertoire of antibodies present in an organism can be indicative of various antigens that the organism has encountered. Such antigens may be derived from external insults, e.g., viral particles or microorganisms such as bacterial cells or fungi. External insults may also be allergens such as pollen or gluten, or environmental factors such as toxins. An organism may also generate antibodies specific to internal antigens. For example, autoimmune disorders are caused by the formation of antibodies that recognize antigens of the host organism. Autoantibodies to various cancer antigens have been observed. In sum, a host organism can comprise antibodies to numerous external and internal antigens indicative of a multitude of diseases, disorders and other environmental factors. Thus, the compositions and methods of the invention can be used to characterize any number of phenotypes in an organism, including without limitation determining environmental exposures and/or providing a diagnosis, prognosis or theranosis for various medical conditions. These conditions include without limitation infectious, autoimmune, parasitic, allergic, neoplastic, genetic, oncological, neurological, cardiovascular, and endocrine diseases and disorders.

### Digital Serology to Determine K-mer Enrichment Scores

As described herein, k-mer scores from each protein of interest are determined by identifying an enrichment score for each k-mer in a protein from a proteome corresponding to a disease or condition from each sample and each cohort. In some embodiments, digital serology is used to determine the k-mer scores from the sera of each sample. Digital Serology is a Next-generation Sequencing (NGS)-based assay similar to other biopanning assays in which peptide libraries are screened with human serum to map human antibody repertoires. The assay involves 4 main steps: 1) incubation of serum with the peptide library and affinity selection of library members expressing peptides that are specific to the antibody repertoire for each serum sample; 2) purification of plasmids that encode these peptides; 3) PCR amplification of the region of the plasmids encoding the peptides (amplicons) and barcoding of each sample with sample-specific primers (allowing samples to be pooled and sequenced together on a single NGS run); and 4) amplicon sequencing by NGS. Once the amplicons are sequenced, the data can be used to identify and determine absolute counts of k-mer sequences identified based on the peptides to which antibodies in the sera from each sample bind. These absolute counts can then be used to determine a score for each k-mer, such as an enrichment score or a comparison score.

Peptide Libraries/Display Libraries

As used herein, a “library of peptides” or a “peptide library” refers to a collection of a peptide fragments typically used for screening purposes. The terms “peptide,” “polypeptide,” “amino acid sequence,” “peptide sequence,” and “protein” are used interchangeably to refer to two or more amino acids linked together and imply no particular length. Amino acids and peptides can be naturally occurring or synthetic (e.g., unnatural amino acids or amino acid analogs). Amino acids and peptides can also comprise, or be further modified to comprise, reactive groups, such as reactive groups for attaching amino acids or peptides to solid substrates, reactive groups for labeling amino acids or peptides, or reactive groups for attaching other moieties of interest to amino acids or peptides. Reactive groups include, but are not limited to, chemically-reactive groups such as reactive thiols (e.g., maleimide based reactive groups), reactive amines (e.g., N-hydroxysuccinimide based reactive groups), “click chemistry” groups (e.g., reactive alkyne groups), and aldehydes bearing formylglycine (FGly).

In general, a peptide library contains a large variety of unique peptides. For example, the diversity of the library (sometimes referred to as “complexity” of the library) can be more than 10⁴, more than 10⁵, more than 10⁶, more than 10⁷, more than 10⁸, more than 10⁹, more than 10¹⁰, or more than 10¹¹ unique peptides. The library can be a random peptide library where the amino acid sequences are unbiased. A particular embodiment of a random/unbiased library is one constructed to represent all possible amino acid sequences of designated length(s).

A peptide library can also be a non-random library where the amino acid sequences are biased in their representation. For example, a library can be biased to represent, over represent, predominantly represent, or only represent amino acid sequences characteristic of a particular feature, such as epitopes or antigens associated with a particular disease (e.g., a bacterial infection, a viral infection, a parasitic infection, an autoimmune disorder, cancer, allergies etc.), condition, species (e.g., mammal, human, bacteria, virus etc.), protein, class of proteins, protein motif (e.g., phosphorylation motifs, binding motifs, protein domains, etc.), amino acid property (e.g., hydrophobic, hydrophilic, acidic, basic, or steric amino acid properties), or any other subset of amino acid sequences that is rationally designed. A library can be biased to also avoid certain amino acid sequences or motifs.

A peptide library can also combine the features of a non-random and random peptide library. For example, one or more select positions within an amino acid sequence may be a constant amino acid and other positions within the sequence may be fully random or biased based on other properties. In other examples, one or more select positions within an amino acid sequence may be selected from a defined subset of amino acids. One skilled in the art will appreciate that the various biases described can combined to achieve a desired purpose of the peptide library, such as a targeted screen.

Typically, peptides in a library can also all fall within a range of lengths. For example, the peptides in a library may be different lengths, but all fall within a defined range of lengths. The selected range can be any length useful for the present invention, such as any length suitable for displaying an epitope sequence capable of recognition by a binding molecule. The peptides in a library can be at least 5, 6, 7, 8, 9, 10, 11, 12, 13, 14, 15, 16, 17, 18, 19, or 20 amino acids in length. The peptides in a library can also be 5-30, 5-25, 5-20, 5-15, 5-10, 10-30, 10-25, 10-20, or 10-15 amino acids in length. The peptides in a library can also be 7-14, 8-14, 9-14, 10-14, 11-14, 12-14, 7-13, 8-13, 9-13, 10-13, 11-13, 12-13, 7-12, 8-12, 9-12, 10-12, 11-12, 7-11, 8-11, 9-11, or amino acids in length. If desired, the peptides in the library can also be greater than 30, greater than 40, greater than 50, greater than 75, greater than 100, greater than 200, or greater than 300 amino acids in length.

Peptides in a library can also be an identical defined length, i.e., all the peptides in the library have the same number of amino acids. The defined length can be any length useful for the present invention, such as any length suitable for displaying an epitope sequence capable of recognition by a binding molecule. The defined length can be 5, 6, 7, 8, 9, 10, 11, 12, 13, 14, 15, 16, 17, 18, 19, or 20 amino acids in length.

A peptide expression library refers to a collection of nucleic acid sequences capable of expressing a peptide library. The nucleic acid sequences can be constructed to achieve a desired library property including those described above, such as peptide diversity, peptide randomization or biasing, and/or peptide length. Any suitable nucleic acid allowing expression of the peptides of interest may be used. In general, the nucleic acid will be a vector. As used herein, a “vector” refers to nucleic acid construct capable of directing the expression of a gene of interest, typically in a host organism, such as a bacterial cell, mammalian cell, or bacteriophage. A vector typically contains the appropriate transcriptional and translational regulatory nucleotide sequences recognized by the desired host for peptide expression, such as promoter sequences. A promoter sequence can be a constitutive promoter. A promoter sequence can be an inducible promoter, where transcription of the encoded sequences is induced by addition of an analyte, chemical, or other molecule, such as a Tet-on system. A variation of an inducible promoter system is a system where transcription is actively repressed, and addition of an analyte, chemical, or other molecule removes the repression, such as addition of arabinose for an arabinose operon promoter or a Tet-off system. A vector can also include elements that facilitate vector construction and production, such as restriction sites, sequences that direct vector replication, drug selection genes or other selectable markers, and any other elements useful for cloning and library production. A typical vector can be a double stranded DNA plasmid in which the nucleic acid sequences encoding the desired peptides is inserted using standard cloning techniques in a location and orientation capable of directing peptide expression. Other vectors include, but are not limited to, nucleic acid constructs useful for in vitro transcription and translation, linear nucleic acid constructs, and single-stranded DNA or RNA nucleic acid constructs.

In general, the number of copies of a specific nucleic acid sequence for each of the candidate peptides is present at a roughly equivalent number, though some variation in number may occur due to probability. A typical peptide expression library can contain more than one copy of a specific nucleic acid sequence (e.g., multiple copies of the same vector). However, in examples where a plurality of samples each contain members of the peptide expression library, the absolute number of each of the candidate peptides may not be equivalent between samples. For example, zero or one copy of a specific nucleic acid sequence can be present in a given sample while one or more copies may be present in another given sample. While the number of copies of a specific nucleic acid sequence need not be identical to the number of copies of other specific nucleic acid sequences, it is generally assumed that about the same number of sequences are present for each of the candidate peptides.

Peptide expression libraries include, but are not limited to, bacterial expression libraries, yeast expression libraries, bacteriophage expression libraries, and mammalian expression libraries. Particular peptide libraries and peptide expression libraries useful for the present invention are described in more detail in issued U.S. Pat. No. 7,256,038, issued U.S. Pat. No. 8,293,685, issued U.S. Pat. No. 7,612,019, issued U.S. Pat. No. 8,361,933, issued U.S. Pat. No. 9,134,309, issued U.S. Pat. No. 9,062,107, issued U.S. Pat. No. 9,695,415, and U.S. Patent Application Publication US 2016/0032279, each herein incorporated by reference in its entirety.

Unique Nucleic Acid Sequences

As used herein, a “unique nucleic acid sequence” refers to a defined unique nucleic acid sequence specific for a given control vector expressing a control binding target. In general, while more than one control vector within a peptide expression library can express the same control binding target, a defined control vector (including multiple copies thereof) contains an identical unique nucleic acid sequence. The peptide expression library can contain one, two, three or more specific control vectors (e.g., one, two, three or more defined subsets where each subset contains an identical unique nucleic acid sequence).

The unique nucleic acid sequences can be at least 4, 5, 6, 7, 8, 9, 10, 11, 12, 13, 14, 15, 16, 17, 18, 19, 20, 21, 22, 23, 24, 25, 26, 27, 28, 29, or 30 nucleotides in length. In examples where the peptide expression library contains two or more control vectors, each unique nucleic acid sequences can be an identical defined length, such as 4, 5, 6, 7, 8, 9, 10, 11, 12, 13, 14, 15, 16, 17, 18, 19, 20, 21, 22, 23, 24, 25, 26, 27, 28, 29, or 30 nucleotides in length. In examples where the peptide expression library contains two or more control vectors, each of the unique nucleic acid sequences can differ by at least 2, at least 3, at least 4, at least 5, at least 6, at least 7, at least 8, at least 9, at least 10-15, at least 15-20, or at least 20-30 nucleotides.

Unique nucleic acid sequences can be in a portion of the control vector such that it is not transcribed but is in a region constructed to allow amplification for downstream processes, such as NGS. Unique nucleic acid sequences can encode a unique peptide sequence expressed a part of the defined peptide sequence.

Unique Peptide Sequences

Unique nucleic acid sequences can encode a unique peptide sequence expressed a part of the defined peptide sequence. The unique peptide sequences can be at least 5, 6, 7, 8, 9, 10, 11, 12, 13, 14, 15, 16, 17, 18, 19, or 20 amino acids in length. In examples where the peptide expression library contains two or more control vectors, each unique peptide sequences can be an identical defined length, such as 5, 6, 7, 8, 9, 10, 11, 12, 13, 14, 15, 16, 17, 18, 19, or 20 amino acids in length. Defined peptide sequences and unique peptide sequences can be immediately adjacent to each other or separated by an additional peptide sequence, and can be N-terminal or C-terminal of the unique peptide sequence.

Composition of the defined peptide sequence, when expressed, can be important to control. For example, in examples where the peptide expression library contains two or more control vectors, the various defined peptide sequence can be constructed to limit the potential effect of amino acid composition on overall expression that may lead to artifacts. In a non-limiting illustrative example, each of the defined peptides each are composed overall of the same amino acids but the order of the amino acids is unique for each defined peptide. Thus, any potential expression bias due to presence of a particular amino acid will be minimized. In other examples, at least one amino acid in the overall composition is different but is substituted for an amino acid of the same class, e.g., hydrophobic, hydrophilic, etc.

Library Array

In a series of embodiments, a composition can be composed of two or more of the peptide expression library compositions described above. The two or more peptide expression library compositions can each be contained in a separate container, such as a well in a multi-well plate, a microcentrifuge tube, a test tube, a tube, and a PCR tube. Each of the separate containers can comprise the same library of nucleic acid sequences encoding the library of peptides but where each container contains a different control vector (i.e., a control vector with a unique nucleic acid sequence). In another example, each of the separate containers can comprise the same library of nucleic acid sequences encoding the library of peptides but where each container contains a different combination of control vectors, e.g., where a given container may share one or more of the control vectors in common with another container, but the exact combination of control vectors is unique to that given container. The combination of control vectors can also be such that a given container does not share any of the control vectors with another container.

In a particular embodiment, a container can be a well within a multi-well plate, e.g., a 96-well plate, and the compositions are arranged such that each of the peptide expression library compositions contains at least one control vector that is different than those in an adjacent well. In another particular embodiment, a container can be a well within a multi-well plate, each of the peptide expression library compositions contains at least two vector controls, and the compositions are arranged such that each adjacent well does not share a control vector in common.

The collection of peptide expression library compositions can be 2, 3, 4, 5, 6, 7, 8, 9, 10-15, 16-24, 24-48, 48-96, or 96-384 peptide expression library compositions. The collection of peptide expression library compositions can be at least 10, at least 20, at least 50, at least 100, at least 200, at least 300, at least 500, at least 1000, or at least 2000 expression library compositions.

Array Surfaces

As used herein, “array surfaces” refers to any surface that can be configured to display (i.e., present) binding targets in a manner suitable for recognition by their respective binding molecules.

Array surfaces can be biological surfaces (e.g., the outer membrane surface of cell). Biological entities that can be used include, but are not limited to, a mammalian cell, a yeast, a bacteria, a virus, and a bacteriophage. The members of the library of peptides (e.g., candidate peptides) and/or the control binding targets can be engineered to be expressed on the surface of a cell, such as constructing the library of nucleic acid sequences encoding the library of peptides or the nucleic acid sequences encoding the control binding targets to also encode a cell surface display peptide sequence configured to be expressed as part of the peptide and capable of directing the peptides for display on the biological entity surface. Illustrative non-limiting examples of E. coli cell surface displayed libraries are described in greater detail in in issued U.S. Pat. No. 7,256,038, issued U.S. Pat. No. 8,293,685, issued U.S. Pat. No. 7,612,019, issued U.S. Pat. No. 8,361,933, issued U.S. Pat. No. 9,134,309, issued U.S. Pat. No. 9,062,107, issued U.S. Pat. No. 9,695,415, and U.S. published application US20160032279, each herein incorporated by reference for all it teaches.

Array surfaces can include solid supports. Solid supports can be have proteins, nucleic acids, or both attached to their surface and can be adapted for use in the present invention. Methods of attaching proteins and nucleic acids are known to those skilled in the art and include, but are not limited to, use of chemically reactive groups such as reactive thiols (e.g., maleimide based reactive groups), reactive amines (e.g., N-hydroxysuccinimide based reactive groups), “click chemistry” groups (e.g., reactive alkyne groups), aldehydes bearing formylglycine (FGly) and other cognate modifications (e.g., biotin-streptavidin pairs, disulfide linkages, polyhistidine-nickel).

In general, the array surface used will be the same for both the library of peptides and the control binding targets. The array surfaces used for the library of peptides can be different from the control binding targets, if desired.

Assay Methods

As used herein, “contacting” refers to any method of bringing the specimen binding molecules and the control binding molecules in proximity to and under conditions sufficient for binding to their respective binding targets. The contacting of the different components can be performed in any suitable order. For example, the peptide expression library composition and the control binding molecule can be contacted prior to contacting either with the sample. In another example, the sample and the control binding molecule can be contacted prior to contacting either with the peptide expression library composition.

Contacting can include mixing all the compositions together. Mixing can be performed in a container, such as a well in a multi-well plate, a microcentrifuge tube, a test tube, a tube, and a PCR tube. Mixing can include rotating, incubating, pipetting, inverting, vortexing, shaking, or otherwise mechanically disturbing components.

Isolation steps used herein can be any method useful for retrieving specimen and control binding molecules. Isolation can involve the use of capture entities. Isolation methods include, but are not limited to magnetic isolation, bead centrifugation, resin centrifugation, and FACS. A particular isolation method can be selected based on the properties of a capture entity, if used, for example magnetic isolation of magnetic beads or FACS isolation of fluorescent beads.

Determining steps, as used herein, in general can use any method for sequencing and/or quantifying nucleic acid, such next generation sequencing (NGS) or quantitative polymerase chain reaction (qPCR). Examples of NGS technologies include massively parallel sequencing techniques and platforms, such as Illumina HiSeq or MiSeq, Thermo PGM or Proton, the Pac Bio RS II or Sequel, Qiagen's Gene Reader, and the Oxford Nanopore MinION. Additional similar current massively parallel sequencing technologies can be used, as well as future generations of these technologies. In some embodiments, the determining step contains the steps of 1) purifying the nucleotide from the biological entity; 2) amplifying the unique nucleic acid sequences and optionally the nucleic acid sequences encoding a peptide bound by the isolated specimen binding molecules; and 2) sequencing the amplified nucleotides. The nucleic acid to be sequenced can also be further modified or processed to facilitate sequencing. For example, nucleic acid can be modified for multiplexed high-throughput sequencing of multiple samples simultaneously, such as adding a sample identifying nucleic acid sequence unique to the sample to terminus of the amplified nucleotides during the amplification step.

Various nucleic acid sequences (e.g., sequences encoding a library of peptides, sequences encoding a control binding target, unique nucleic acid sequences) can be differentiated from each other during the determining step(s). Differentiating various nucleic acid sequences includes differentiating portions of nucleic acid sequences, such as differentiating the different sequences in a vector (e.g., differentiating a nucleic acid sequence encoding a binding target from unique nucleic acid sequence). Sequences can be differentiated based on specific characteristics, such as position within a sequence, identity of adjacent sequences, known identity of sequences, or combinations thereof. Sequence alignment algorithms, such as those known in the art, can be used to identify, quantify, and differentiate the different sequences

Enrichment Assessment

The identity and quantity of isolated unique nucleic acid sequences that encode candidate peptides in a peptide expression library can be used to assess the enrichment of peptide sequences in a sample.

The assessment can involve the use of a computer. In general, a computer is adapted to execute a computer program for providing results, for example the results of determining nucleic acid sequences such as those sequences produced during a sequencing step or the results of an assessment step providing enrichment results from a sample. Generally, the steps of determining the nucleic acid sequences and determining enrichment involve such a large number of computations, particularly given the number of sequences generally under consideration, that they are carried out by a computer system in order to be completed in a reasonable amount of time. They cannot be practically carried out by the human mind or by pen and paper alone.

A computer can include at least one processor coupled to a chipset. Also coupled to the chipset can be a memory device, a memory controller hub, an input/output (I/O) controller hub, and/or a graphics adaptor. Various embodiments of the invention may be implemented as a computer program instructions stored in a non-transitory computer readable storage medium for execution by a processor of a computer system. The instructions define functions of the embodiments (including the methods described herein). Illustrative computer-readable storage media include, but are not limited to: (i) non-writable storage media (e.g., read-only memory devices within a computer such as CD-ROM disks readable by a CD-ROM drive, flash memory, ROM chips or any type of solid-state non-volatile semiconductor memory) on which information is permanently stored; and (ii) writable storage media (e.g., floppy disks within a diskette drive or hard-disk drive or any type of solid-state random-access semiconductor memory) on which alterable information is stored.

A computer can include a means for programming the computer (i.e., providing computer program instructions), such as providing sequence alignment software or quality control assessment software. A computer can include a means for inputting information, such as sequences, including, but not limited to, a keyboard, a mouse, a touch-screen interface, or combinations thereof. A computer can include a means to display information and images, such as a graphics adaptor and display. A computer can include means to connect to other computers (e.g., computer networks), such as a network adaptor.

Some portions of the description herein describe the embodiments in terms of algorithms and symbolic representations of operations on information. These algorithmic descriptions and representations are commonly used by those skilled in the data processing arts to convey the substance of their work effectively to others skilled in the art. These operations, while described functionally, computationally, or logically, are understood to be implemented by computer programs, equivalent electrical circuits, or the like. The described operations and their associated modules may be embodied in software, firmware, hardware, or any combinations thereof.

An enrichment can be a ratio or percentage of unique peptide sequences specific present in a sample.

In one example, the determining step, such as NGS to identify and quantify all unique nucleic acid sequences, can be used to calculate a percentage of the unique nucleic acid sequences specific for the sample (i.e., the sequence(s) assigned to a given sample) present relative to a total number of unique nucleic acid sequences, wherein the total number comprises the number of the unique nucleic acid sequences specific for the sample and the number of the unique nucleic acid sequences not specific for the sample (i.e., the quantity of all unique nucleic acid sequences regardless of sample assignment). A percentage that falls below an established quality control standard can indicate an error in the method, such as contamination between samples, and invalidate the sample. The quality control standard can be between 90-100%, between 92-100%, between 95-100%, between 96-100%, or between 98-100%. The quality control standard can be about 90%, about 92%, about 95%, about 96%, about 97%, about 98%, or about 99%. The quality control standard can be at least 98%.

In another example, the determining step, such as NGS to identify and quantify all unique nucleic acid sequences, can be used to calculate a percentage of the unique nucleic acid sequences specific for the sample relative to a total number of nucleic acid sequences, the total number comprising the number of the unique nucleic acid sequences specific and not specific the sample and the number of nucleic acid sequences encoding the peptides in the library of peptides. A percentage that falls above or below an established quality control standard can indicate an error in the method and invalidate the sample. The quality control standard can be between 0.01%-2.0%, between 0.05%-2.0%, or between 0.01%-1.0%. The quality control standard can between 0.05%-1.0%. A computer, as described herein, can be used to perform determination (e.g., sequencing) and assessment steps described herein.

### Computer

Many of the assays described herein (e.g., k-mer enrichment score determination, k-mer identification in proteins of a proteome, determining antigenic score for each protein in the condition-relevant proteome for each sample from each cohort using k-mer enrichment values, determining outlier antigen scores for each protein, identifying relevant antigens for condition of interest, identifying antigenic motif on an antigen, sequence alignment/clustering, NGS applications, etc.) typically require the use of a computer as they cannot be practically carried out by the human mind or by pen and paper alone. In general, a computer is adapted to execute a computer program for providing results, for example the results of determining nucleic acid sequences such as those sequences produced during a sequencing step or the results of an assessment step providing if the assay meets a quality control standard. Generally, the steps of determining the nucleic acid sequences and determining the results of the assessment step involve such a large number of computations, particularly given the number of sequences generally under consideration, that they are carried out by a computer system in order to be completed in a reasonable amount of time. They cannot be practically carried out by the human mind or by pen and paper alone. A computer can include at least one processor coupled to a chipset. Also coupled to the chipset can be a memory device, a memory controller hub, an input/output (I/O) controller hub, and/or a graphics adaptor. Various embodiments of the invention may be implemented as a computer program instructions stored in a non-transitory computer readable storage medium for execution by a processor of a computer system. The instructions define functions of the embodiments (including the methods described herein). Illustrative computer-readable storage media include, but are not limited to: (i) non-writable storage media (e.g., read-only memory devices within a computer such as CD-ROM disks readable by a CD-ROM drive, flash memory, ROM chips or any type of solid-state non-volatile semiconductor memory) on which information is permanently stored; and (ii) writable storage media (e.g., floppy disks within a diskette drive or hard-disk drive or any type of solid-state random-access semiconductor memory) on which alterable information is stored.

A computer can include a means for programming the computer (i.e., providing computer program instructions), such as providing sequence alignment software or quality control assessment software. A computer can include a means for inputting information, such as sequences, including, but not limited to, a keyboard, a mouse, a touch-screen interface, or combinations thereof. A computer can include a means to display information and images, such as a graphics adaptor and display. A computer can include means to connect to other computers (e.g., computer networks), such as a network adaptor. Some portions of the description herein describe the embodiments in terms of algorithms and symbolic representations of operations on information. These algorithmic descriptions and representations are commonly used by those skilled in the data processing arts to convey the substance of their work effectively to others skilled in the art. These operations, while described functionally, computationally, or logically, are understood to be implemented by computer programs, equivalent electrical circuits, or the like. The described operations and their associated modules may be embodied in software, firmware, hardware, or any combinations thereof.

A computer can be used to perform the methods of identifying sample and/or cohort specific antigenic sequences and methods of epitope identification using k-mer enrichment scores, as described herein. In some embodiments, the k-mer level statistics or antigenic peptide information from each sera sample is stored in an efficient database (i.e. BigTable).

The different methods described herein are not mutually exclusive.

### Equivalents and Scope

Those skilled in the art will recognize, or be able to ascertain using no more than routine experimentation, many equivalents to the specific embodiments in accordance with the invention described herein. The scope of the present invention is not intended to be limited to the above Description, but rather is as set forth in the appended claims.

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

It is also noted that the term “comprising” is intended to be open and permits but does not require the inclusion of additional elements or steps. When the term “comprising” is used herein, the term “consisting of” is thus also encompassed and disclosed.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise.

All cited sources, for example, references, publications, databases, database entries, and art cited herein, are incorporated into this application by reference, even if not expressly stated in the citation. In case of conflicting statements of a cited source and the instant application, the statement in the instant application shall control.

Section and table headings are not intended to be limiting.

### Examples

Below are examples of specific embodiments for carrying out the present invention. The examples are offered for illustrative purposes only, and are not intended to limit the scope of the present invention in any way. Efforts have been made to ensure accuracy with respect to numbers used (e.g., amounts, temperatures, etc.), but some experimental error and deviation should, of course, be allowed for.

The practice of the present invention will employ, unless otherwise indicated, conventional methods of protein chemistry, biochemistry, recombinant DNA techniques and pharmacology, within the skill of the art. Such techniques are explained fully in the literature. See, e.g., T. E. Creighton, Proteins: Structures and Molecular Properties (W.H. Freeman and Company, 1993); A. L. Lehninger, Biochemistry (Worth Publishers, Inc., current addition); Sambrook, et al., Molecular Cloning: A Laboratory Manual (2nd Edition, 1989); Methods In Enzymology (S. Colowick and N. Kaplan eds., Academic Press, Inc.); Remington's Pharmaceutical Sciences, 18th Edition (Easton, Pa.: Mack Publishing Company, 1990); Carey and Sundberg Advanced Organic Chemistry 3rd Ed. (Plenum Press) Vols A and B(1992)

Methods useful for the present invention, e.g., digital serology including motif determination and motif analysis, are also described in more detail in Pantazes, et al. and International PCT Patent Application Publication WO2017/083874A1, each herein incorporated by reference for all they teach.

### Example 1: Digital Serology Assay

Bacterial Surface Display Antibody Screen

A large, high-quality, bacterial-display, random, 12-mer peptide library composed of 8×10⁹ independent transformants, was constructed using trinucleotide oligos to eliminate stop codons and normalize amino acid usage frequencies. The 12-mer peptide library was displayed on E. coli via the N-terminus of a previously reported, engineered protein scaffold (eCPX), as described in more detail in Rice, et al., herein incorporated by reference for all it teaches. Vectors, methods, and other tools useful in the E. coli surface displayed peptide library are described in more detail in issued U.S. Pat. No. 7,256,038, issued U.S. Pat. No. 8,293,685, issued U.S. Pat. No. 7,612,019, issued U.S. Pat. No. 8,361,933, issued U.S. Pat. No. 9,134,309, issued U.S. Pat. No. 9,062,107, issued U.S. Pat. No. 9,695,415, and U.S. published application US20160032279, each herein incorporated by reference for all they teach.

To remove E. coli binding antibodies from serum samples prior to library screening, an induced culture of cells expressing the library scaffold alone was incubated with diluted sera (E. coli strain MC1061 [FaraΔ 139 D(ara-leu)7696 GalE15 GalK16 Δ (lac)X74 rpsL (StrR) hsdR2 (rK−mK+) mcrA mcrB1] was used with surface display vector pB33eCPX). eCPX cultures grown overnight at 37° C. with vigorous shaking (250 rpm) in LB (10 g tryptone, 5 g yeast extract, 10 g/L NaCl) supplemented with 34 μg/mL chloramphenicol (CM) and 0.2% glucose were collected by centrifugation, inoculated in fresh LB+CM, grown to an OD₆₀₀=0.6, and induced for 1 hour at 37° C. with 0.02% wt/vol L(+)-arabinose. After induction, cells were centrifuged at 3,000 relative centrifugal force (rcf) for 5 min., washed once with cold PBST (PBS+0.1% Tween 20), and resuspended in 750 μL PBST containing serum diluted 1:25 (1×10¹⁰ cells per depletion sample). Samples were incubated overnight at 4° C. with gentle mixing on an orbital shaker (20 rpm). Antibodies that bound to E. coli or the eCPX scaffold were removed by centrifugation of the incubated culture at 5,000 rcf for 5 min. twice, recovering the serum supernatant after each centrifugation. Depleted serum was stored at 4° C. for up to 2 weeks during use.

The bacterial display peptide library was used to screen and isolate peptide binders to antibodies in individual serum samples through Magnetic Activated Cell Sorting (MACS). The MACS screen employed magnetic selection to enrich the library for antibody binding peptides as well as reduce the library size suitable for the subsequent screening steps. A frozen aliquot of the library containing 10¹¹ cells (>10× the expected diversity) was thawed and inoculated into 500 ml LB+CM. After growth to an OD₆₀₀=0.6 at 37° C. with 250 rpm shaking, the cells are induced with 0.02% wt/vol L(+)-arabinose for one hour using the same growth conditions. Cells (5×10¹⁰ per sample) were collected by centrifugation (3,000 rcf for 10 min.) and resuspended in 750 μL cold PBST. Prior to incubation with serum, cells were cleared of peptides that bind protein A/G by incubating cells with washed protein A/G magnetic beads (Pierce) at a ratio of one bead per 50 cells for 45 min. at 4° C. with gentle mixing. Magnetic separation for 5 min. (×2) was used to recover the unbound cells. Recovered cells from the supernatant are centrifuged, resuspended in diluted sera (1:25) and incubated for 45 min. at 4° C. with gentle mixing. Following serum incubation, cells were washed by centrifugation and resuspended in 750 μL cold PBST (×3). After the final resuspension, washed protein A/G magnetic beads were added at a ratio of one bead per 50 cells. After a 45 min. incubation with protein A/G beads at 4° C. with gentle mixing, a second magnetic separation isolated cells expressing peptides that bind to serum antibodies. The supernatant (unbound cells) was discarded and the separated cells/beads were washed with 750 μL cold PBST. 5 repeat washes were performed while the tube was being magnetized. After the last wash, the beads were resuspended in 1 mL of LB and inoculated into 25 mL LB+CM+glucose to suppress expression. The flask was grown overnight at 37° C. with shaking at 250 rpm.

Next Generation Sequencing

Cells grown overnight after the MACS enrichment were collected and plasmid was extracted using a plasmid miniprep kit (Qiagen). The random peptide region was amplified using a two-step PCR. For the first PCR step, the primers include adaptors specific to the Illumina sequencing platform with annealing regions that flank the random region (peptide library) of the eCPX scaffold. Bolded regions anneal to the eCPX scaffold, and nnnnn are 5 random degenerate bases that help the NGS protocol discriminate sequencing reads on the sequencing chip, particularly those sequences with a constant vector sequence ahead of the peptide encoding nucleotides.

Forward Primer (SEQ ID NO: 1):
TCGTCGGCAGCGTCAGATGTGTATAAGAGACAGnnnCCAGTCTGGCCAG
GG
Reverse Primer (SEQ ID NO: 2):
CCAGTACTACGGCATCACTGCTGTCTCTTATACACATCTCCGAGCCCAC
GAGAC

Products from the first PCR were purified after 25 rounds of PCR amplification (touchdown PCR) using Agencourt Ampure XP (Beckman Coulter) clean up beads. Resulting product was subjected to a second round of PCR using Illumina Nextera XT indexing primers (Illumina). These primers provide unique 8 base pair indicies on the 3 prime and 5 prime ends of the amplicons for tracking the sequences back to the sample used for screening and amplicon preparation. Amplicons were cleaned up as before after 8 rounds of PCR amplification (70° C. annealing temp). The final PCR product (amplicon) DNA concentration was measured using DNA high sensitivity reagent on a Qbit instrument (Life Technologies). All samples were normalized to 4 nM and pooled together into a sequencing library.

The pooled sample was diluted and loaded on to the NextSeq instrument. A 75 cycle high-output flow cell was used with single read (one direction) and dual indexing (both 5 prime and 3 prime indicies are sequenced). After sequencing was complete, the samples were automatically de-multiplexed using imputed sample identities with Illumina Nextera XT indicies.

Following NGS analysis, samples were analyzed for enrichment for each k-mer within each 12-mer peptide. Each 12-mer peptide was broken into constitutive k-mer sequences of 5 amino acids (i.e., 5-mer peptide sequences) and 6 amino acids (i.e., 6-mer peptide sequences). For example, the 12-mer protein sequence ABCDEFGHIJKL would be broken into the following 5aa k-mer sequences (i.e., 5-mers): ABCDE, BCDEF, CDEFG, DEFGH, EFGHI, FGHIJ, GHIJK, and HIJKL. The enrichment score was calculated by dividing the number of observed instances (across all 12-mers) for each k-mer by the number of expected instances. Specifically, a z-score for each k-mer was calculated where each z-score indicates the enrichment value minus the mean enrichment for all samples divided by the standard deviation of all samples. This was performed as described in the section “Enrichment Score Calculation” above.

For each protein in each sample, an antigenic score was calculated using k-mer enrichment score for each k-mer in the protein. For the examples provided below, unless otherwise stated, the maximum k-mer enrichment score for each protein was used to determine the antigenic score. This was performed as described under the section “Antigenic Score Calculation” above.

The antigenic score for each protein from the sample cohort and the control cohort was compared to identify the proteins most likely to have high antigenicity specific to the disease or condition of interest. This was performed as described under the section “Cohort Comparison Statistics and Antigen Outlier Score” above.

### Example 2: Discovery of Disease Biomarkers in Cancer Patients Using Protein Level IWAS

Here we provide specific examples of use of the immune wide association method described herein to identify disease-specific, proteome-based, antigenic signals. In this example, serum from patients having melanoma or not having melanoma were obtained and compared using the methods described in Example 1 to identify antigens corresponding to melanoma.

Specifically, 204 serum samples from patients diagnosed as having melanoma were provided for the disease cohort. 6,382 serum samples from patients not known to have cancer were provided for the control cohort. These samples were assayed using the 12-mer peptide library bacterial surface display antibody screen, and enriched cells were sequences using next-generation sequencing. Enrichment for 5-mer and 6-mer k-mers was determined for each sample and compared with protein sequences from a proteome corresponding with melanoma patients to identify antigenic scores. Antigenic scores for each cohort were then compared to identify outlier proteins that exceeded a threshold value to indicate that the protein has a high antigenicity specific for melanoma.

Using this method, we detected several shared antigens that were specific for melanoma patients, including the well-established NY-ESO-1 (cancer/testis) antigen.

We repeated this method for prostate cancer. Specifically, 148 serum samples were taken from 70 patients total at different stages of the disease. A non-cancer control cohort comprising 6,439 active IgG samples was used for comparison to identify prostate cancer-specific antigens as described in Example 1. Our method identified several new candidate antigens, including previously validated antigen NY-ESO-1.

ELISA Validation

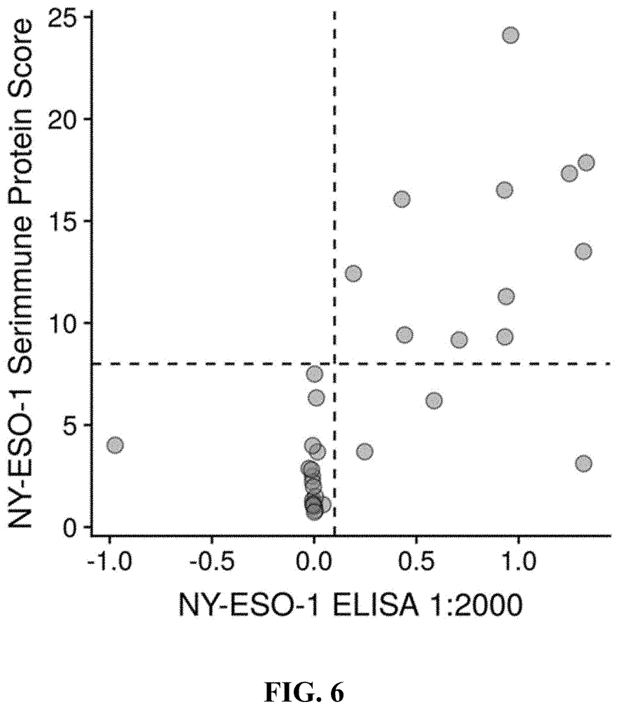

To confirm overall concordance between the method provided herein and a traditional enzyme linked immunosorbant assays (ELISA), we measured the antigenicity of the NY-ESO-1 protein against sera from individual melanoma patients using both ELISA and our method. As shown in FIG. 6, a significant number of melanoma samples showed a specific antigenic response to the NY-ESO-1 protein as determined by both our assay and by ELISA.

### Example 3: Epitope-Level Resolution of Antigenicity of NY-ESO-1 Antigen in Serum from Melanoma Patients

In addition to identifying antigenic proteins corresponding to a disease or condition, we can also use the methods described herein to provide resolution at the epitope level, identifying an established antigenic epitope in the cancer-specific antigen NY-ESO-1.

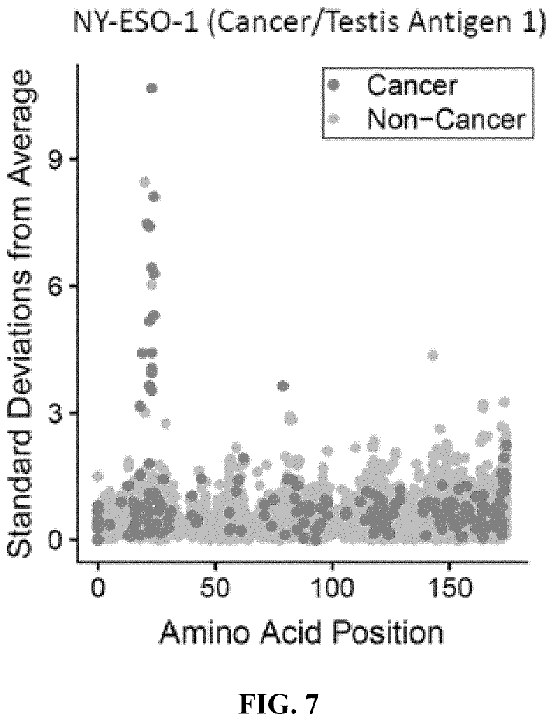

Specifically, the k-mer peptide within NY-ESO-1 with the highest enrichment score from each sample from each cohort was identified as described herein. The S.D. from average and the position within NY-ESO-1 was determined for these k-mer peptides (one per sample) and plotted in FIG. 7. As shown in FIG. 7, cancer patients show a significant and specific antigenic epitope in NY-ESO-1.

This epitope corresponds to a previously identified B-cell epitope in multiple cancers, including melanoma and prostate cancer (see, e.g., Zeng et al., “Dominant B cell epitope from NY-ESO-1 recognized by sera from a wide spectrum of cancer patients: implications as a potential biomarker,” Int J Cancer. 2005; 114:268-273). Therefore, our methods enable identification of both i) novel antigens that correspond to a condition of interest, and ii) one or more epitopes of interest for the antigen by providing high-resolution maps of one or more antigenic regions of interest for the cohort of interest.

### Example 4: Monitoring Epitope Antigenicity for a Disease-Specific Antigen Over a Course of Treatment

Identification of a patient condition can extend to many conditions and phenotypes beyond diagnosis of a disease or disorder. For example, the method provided herein can be used to further subtype patients.

As shown in this example, for any given protein, antigenic epitopes can be identified before and/or after immuno-therapy to predict or monitor a response to therapy.

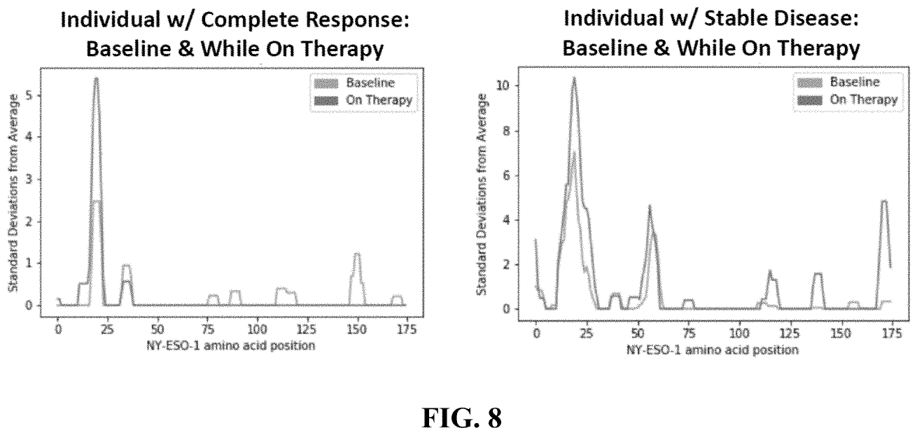

As shown in FIG. 8, epitope-level resolution of antigenicity for NY-ESO-1 was determined from sera of patients i) responsive to therapy and ii) not responsive to therapy both before (‘Baseline’) and after therapy (‘On Therapy’, approximately 3 months after treatment). Distinctions in the high-resolution epitope mapping of NY-ESO-1 from each cohort before and during treatment shows this method can be used to both predict and monitor patient response to therapy.

### Example 5: Discovery of Autoimmunity Biomarkers in Sjogren's Patients Using Protein Level IWAS

As described and shown in the following example, our method can be used to identify antigens specific for an autoimmune condition/disease. Specifically, we identified antigens specific for Sjogren's syndrome.

We performed the k-mer enrichment analysis as described in Example 1 on a disease cohort of 146 samples from patients diagnosed with Sjogren's syndrome and a control cohort of 7,150 samples from patients without known autoimmune disease.

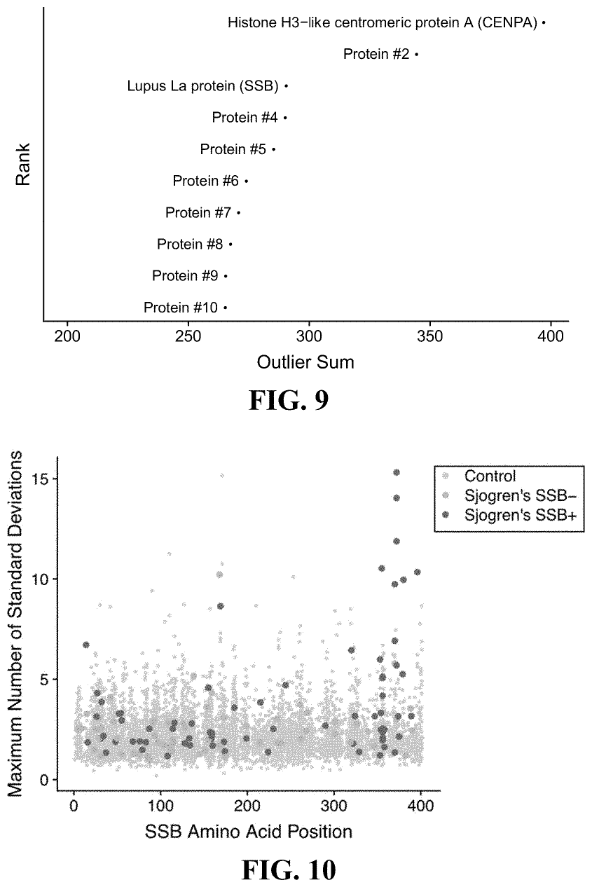

We compared the enrichment scores for k-mer sequences within the human proteome to determine antigenic scores for proteins in the human proteome to identify autoantigens specific for Sjogren's syndrome (as described herein and in Example 1). Autoantigens CENPA and La/SSB, which have been established as corresponding to Sjogren's syndrome, were identified, thereby further validating our method for discovery of autoantigens. Results are shown in FIG. 9.

### Example 6: Epitope-Level Resolution of Antigenicity of SSB Antigen in Sjogren's Patients

As described in Example 3, we determined epitope level-resolution of antigenicity of the SSB antigen by identifying the location and score for the most-enriched k-mer for SSB for each sample from each cohort. As shown in FIG. 10, individuals with k-mer peaks (strong SSB responses) are mostly predicate SSB+ patients. These same major epitopes have been identified in independent studies (see, e.g., Tzioufas et al., “Fine specificity of autoantibodies to La/SSB: epitope mapping and characterization.” Clin Exp Immunol. 1997 May; 108(2): 191-198).

Therefore, we have further validated our method, including high resolution mapping of disorder-specific antigens, by identifying established antigenic epitopes in the Sjogren's autoantigen La/SSB

ELISA Validation

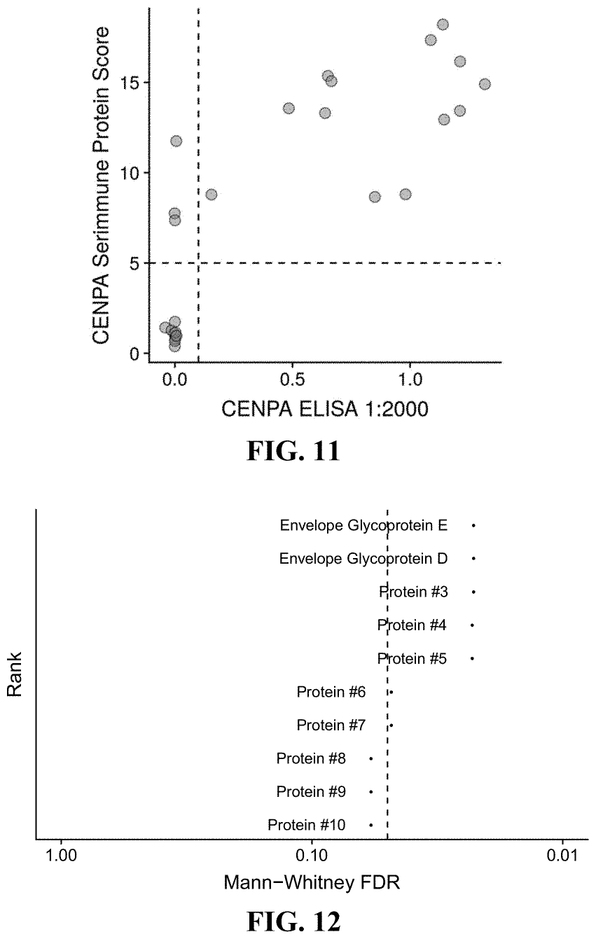

To confirm overall concordance between the method provided herein and a traditional enzyme linked immunosorbant assays (ELISA), we measured the antigenicity of the CENPA protein against sera from Sjogren's patients using both ELISA and our method. As shown in FIG. 11, significant number of samples from Sjogren's patients showed a specific antigenic response to CENPA as determined both by our assay and ELISA. The results therefore show overall concordance of CENPA sample-specific antigenicity between our method and ELISA.

### Example 7: Discovery of Disease Biomarkers for HSV2 Infection Using Protein Level IWAS

As shown in the following example, we also successfully identified disease biomarkers specific for HSV2 infection as compared to vaccination using our method.

In this example, 102 serum samples from patients positive for HSV2 infection (HSV2+/HSV1−) were compared with 14 serum samples from patients 210 days post-HSV2 vaccination

FIG. 12 shows a ranking of antigens specific for the natural HSV2 infection as compared to the HSV2 vaccination. Decreased immune response to Envelope Glycoproteins D and E in vaccine compared to natural infection was identified using our method.

Envelope Glycoprotein E

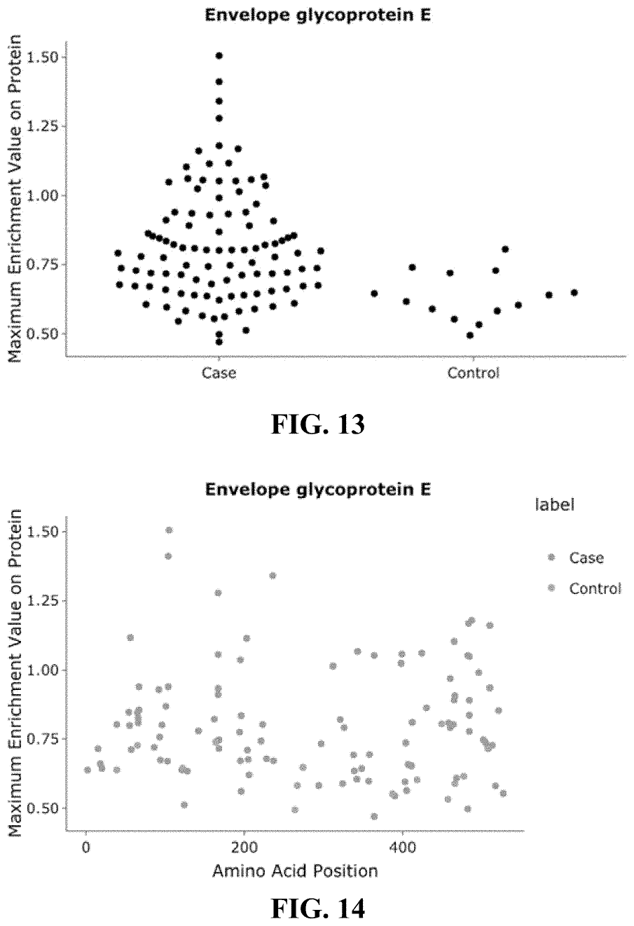

As shown in FIG. 13, maximum enrichment scores for Envelope glycoprotein E k-mers from sera from HSV2 infected patients (‘Case’) (i.e., condition) and HSV2 vaccinated patients (‘Control’) were determined using the methods as described in Example 1 and throughout the specification. These maximum enrichment scores are used here to provide an antigenic score for the glycoprotein.

The most-enriched k-mer for Envelope glycoprotein E from each sample is shown in FIG. 14 by its location and enrichment score for each sample. No conserved epitope is identified. Thus, our method is capable of identifying antigenic proteins specific for a condition, despite a large diversity of identified epitope targets on the protein from samples from the same cohort.

Envelope Glycoprotein D

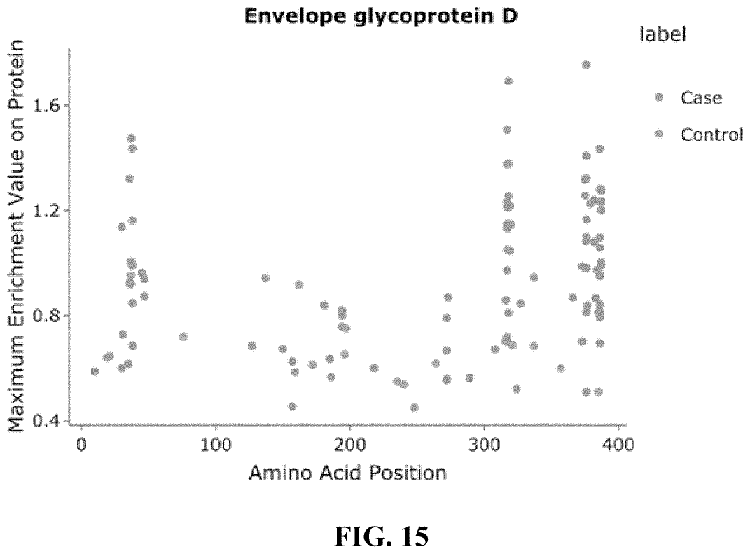

In contrast to Envelope glycoprotein E, high resolution mapping of the antigenic epitopes on Envelope Glycoprotein D using our method shows distinct conserved epitope regions (FIG. 15). However, since there is more than one epitope, our IWAS method provide herein is also preferably suited to identification and characterization of such an antigen.

As shown above, we can successfully discriminate between HSV2 infected and vaccinated samples. We can also identify and characterize with high resolution antigens specific for serum that has been exposed to natural HSV2 infection vs. HSV2 vaccination.

These examples show successful identification of cohort-specific, proteome-based antigenic signals for a variety of conditions, diseases, and phenotypes. This identification is possible even when there is more than one antigenic epitope per antigen for the cohort, or the antigenic epitopes are not conserved across the cohort, and is sensitive enough to discriminate between naturally infected vs. vaccinated subjects.

### Other Embodiments

It is to be understood that the words which have been used are words of description rather than limitation, and that changes may be made within the purview of the appended claims without departing from the true scope and spirit of the invention in its broader aspects.

While the present invention has been described at some length and with some particularity with respect to the several described embodiments, it is not intended that it should be limited to any such particulars or embodiments or any particular embodiment, but it is to be construed with references to the appended claims so as to provide the broadest possible interpretation of such claims in view of the prior art and, therefore, to effectively encompass the intended scope of the invention.

All publications, patent applications, patents, and other references mentioned herein are incorporated by reference in their entirety. In case of conflict, the present specification, including definitions, will control. In addition, section headings, the materials, methods, and examples are illustrative only and not intended to be limiting.

1. A method of identifying an antigen marker for a condition, the method comprising:
identifying a condition cohort and a control cohort for comparison;
providing a set of antigens corresponding to said condition, wherein the sequence of each antigen is tiled into subsequences;
providing an enrichment score for each of said subsequences for both said condition cohort and said control cohort;
for each antigen in said set of antigens:
determining an antigenic score of said antigen for said condition cohort and said control cohort from said enrichment scores for subsequences within said antigen, and
comparing said antigenic score for said condition cohort and said control cohort to determine an antigen outlier score; and
identifying said antigen as an antigen marker for said condition if said antigen outlier score exceeds a threshold value.

2. The method of claim 1, wherein said enrichment score is determined from a motif enrichment score determined for a motif comprising said subsequence.

3. The method of claim 1, wherein said enrichment score is determined from identification of relative binding of subsequences to antibodies from a serum sample between said condition cohort and said control cohort or wherein the method further comprises determining said enrichment score by identifying relative binding of subsequences to antibodies from a serum sample between said condition cohort and said control cohort.

4. (canceled)

5. The method of claim 1, wherein said antigenic score is determined from:
(a) the highest subsequence enrichment score for said antigen sequence in said cohort;
(b) the sum of all subsequence enrichment scores for said antigen sequence in said cohort
(c) the highest average value of subsequence enrichment scores within a window of n subsequences for said antigen sequence in said cohort;
(d) the sum of n maximum subsequence enrichment scores across the antigen sequence.

6. (canceled)

7. (canceled)

8. (canceled)

9. The method of claim 1, wherein said comparing said antigenic score for said condition cohort and said control cohort comprises calculating a statistical difference between antigenic scores from said sample cohort and said control cohort for said antigen.

10. The method of claim 9, wherein said threshold value represents a statistical difference sufficient for identifying said antigen as an antigen marker.

11. The method of claim 9, wherein said statistical difference:
(a) is determined from a statistical analysis selected from the group consisting of: Cohen's d effect size, Mann-Whitney U p-value, Kolmogorov-Smirnov p-value, and Outlier sum; and/or
(b) comprises a correction for multiple hypothesis testing, optionally wherein said correction is Bonferroni correction or false discovery rate.

12. (canceled)

13. (canceled)

14. The method of claim 1, wherein said threshold is determined from a ranking of antigen outlier scores determined from said set of antigens.

15. The method of claim 1, wherein said subsequences are k-mers, wherein said k-mers comprise 5-mers, 6-mers, 7-mers, 8-mers, 9-mers, or 10-mers.

16. (canceled)

17. The method of claim 1, wherein said subsequence comprises a k-mer sequence with at least k-n defined amino acid positions, wherein k is 8, 9 or 10, and wherein n is 2, 3, 4, 5, or 6.

18. The method of claim 1, wherein said antigen sequences are amino acid sequences.

19. The method of claim 1, wherein said antigen marker comprises a protein, a RNA, or an aptamer.

20. The method of claim 1, wherein said condition cohort comprises one or more samples from one or more patients, wherein said patients have been diagnosed with an infection, an autoimmune disorder, a cancer, a neurological disorder, or a chronic disease, or wherein said patient has been administered a therapeutic agent or a vaccine.

21. The method of claim 1, wherein providing said enrichment score comprises:
contacting a display system comprising a plurality of distinct peptides with a biological sample comprising a plurality of antibodies, wherein the plurality of antibodies is known or suspected to comprise antibodies for said condition, and wherein the contacting is performed under conditions sufficient for the specimen antibodies to specifically bind to a cognate epitope on said plurality of distinct peptides;
measuring the binding between the plurality of distinct peptides and the specimen antibodies; and
identifying an enrichment score for said subsequence from the amount of binding measured for said subsequence.

22. The method of claim 21, wherein said peptides are randomly generated.

23. The method of claim 21, wherein said peptides are from 8-mer to 15-mer peptides, optionally wherein said peptides are 12-mer peptides.

24. (canceled)

25. The method of claim 21, said display system comprising at least 10, at least 100, at least 1000, at least 10⁴, at least 10⁵, at least 10⁶, at least 10⁷, or at least 10⁸ distinct peptides.

26. (canceled)

27. The method of claim 1, wherein said determination of said antigenic score and said antigenic outlier score is implemented as a set of computer program instructions stored on a non-transitory computer readable storage medium for execution by a processor of a computer system.

28. The method of claim 1, wherein said identifying said antigen as an antigen marker for said condition if said antigen outlier score exceeds a threshold value is implemented as a set of computer program instructions stored on a non-transitory computer readable storage medium for execution by a processor of a computer system.

29. A method of identifying one or more antigenic epitopes on an antigen marker specific for a condition cohort as compared to a control cohort, the method comprising:
identifying a condition cohort and a control cohort for comparison;
providing an antigen corresponding to said condition, wherein the sequence of said antigen is tiled into subsequences;
providing an enrichment score for each of said subsequences for samples from both said condition cohort and said control cohort;
determining a statistical difference between enrichment scores in one or more regions of said antigen for said samples from said condition cohort compared to said samples from said control cohort; and
identifying said one or more regions as an antigenic epitope specific for said condition cohort as compared to said control cohort if said statistical difference exceeds a threshold value.

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. (canceled)

50. (canceled)

51. (canceled)

52. (canceled)

53. A system for identifying an antigen marker for a condition comprising a non-transitory computer readable storage medium and a processor, said storage medium comprising:
enrichment scores for subsequences of antigens corresponding to said condition, said enrichment scores specific to a condition cohort and a control cohort;
instructions for generating an antigenic score of each antigen specific to said condition cohort and said control cohort from said enrichment scores of subsequences of said antigen; and
instructions for generating an antigenic outlier score by comparing the statistical difference between said antigenic score for said antigen specific for said condition cohort and said control cohort.

54. (canceled)

55. (canceled)

56. (canceled)

57. (canceled)

58. (canceled)

59. (canceled)

60. (canceled)

61. (canceled)

62. (canceled)


SEQUENCE LISTING (3 sequences)

SEQ ID NO: 1 - DNA, 53 bp, Artificial Sequence
  Description of Artificial Sequence Synthetic primer
  modified_base (34)..(38): a, c, t, g, unknown or other
tcgtcggcagcgtcagatgtgtataagagacagnnnnnccagtctggccaggg

SEQ ID NO: 2 - DNA, 54 bp, Artificial Sequence
  Description of Artificial Sequence Synthetic primer
ccagtactacggcatcactgctgtctcttatacacatctccgagcccacgagac

SEQ ID NO: 3 - protein, 8 aa, Artificial Sequence
  Description of Artificial Sequence Synthetic peptide
  MOD_RES (3)..(4): Any amino acid
  MOD_RES (7)..(7): Any amino acid
  MOD_RES (8)..(8): Glu or Asp
QPXXPFXX